(19) United States
(12) Patent Application Publication
(10) Pub. No.: US 2020/0002709 A1
(43) Pub. Date: Jan. 2, 2020

(54) METHODS FOR ALTERING AMINO ACID CONTENT IN PLANTS

(71) Applicant: CELLECTIS, Paris (FR)

(72) Inventors: Song Luo, Chicago, IL (US); Benjamin Clasen, South St. Paul, MN (US); Feng Zhang, Maple Gove, MN (US); Nicholas J. Baltes, Maple Grove, MN (US); Javier Gil Humanes, Circle Pines, MN (US)

(21) Appl. No.: 16/328,323

(22) PCT Filed: Aug. 30, 2017
(86) PCT No.: PCT/IB2017/055216

§ 371 Date: Feb. 26, 2019

Related U.S. Application Data
(60) Provisional application No. 62/382,352, filed on Sep. 1, 2016.
(60) Provisional application No. 62/486,794, filed on Apr. 18, 2017.

(51) Int. Cl. C12N 15/82

(52) U.S. Cl. CPC C12N 15/8213; C12N 15/8254; C12N 15/8253

(57) ABSTRACT

Materials and methods are provided for making plants (e.g., soybean varieties, wheat varieties, or corn varieties) with altered amino acid content. For example, materials and methods are provided for making TALE nuclease-induced mutations in genes encoding seed storage proteins, or by making TALE nuclease-induced deletions of within seed storage protein genes.

### CROSS-REFERENCE TO RELATED APPLICATIONS

This application claims benefit of priority from U.S. Provisional Application Ser. No. 62/382,352, filed on Sep. 1, 2016, and U.S. Provisional Application Ser. No. 62/486,794, filed on Apr. 18, 2017, which are incorporated herein by reference in their entirety.

### TECHNICAL FIELD

This document provides materials and methods for generating plants, plant parts, and plant cells with altered levels of particular amino acids, including by through reducing the levels of certain seed storage proteins.

### BACKGROUND

Humans and some other animals (e.g., farm animals) are unable to synthesize several amino acids that are required for survival, including histidine, isoleucine, leucine, methionine, phenylalanine, threonine, tryptophan, valine, and lysine. As a result, the diet of humans and farm animals must contain sufficient levels of these essential amino acids. In developed countries, optimal levels of essential amino acids are generally achieved through diets consisting of meat, eggs, milk, cereals, and legumes. In developing countries, however, diets are frequently restricted to major crop plants, which can result in a deficiency of particular amino acids. For example, soybean (Glycine max L. Merr.) is an important source of protein for livestock, and is of growing importance as a protein source for human consumption. Although soybean has the highest protein content among seed crops, the protein quality tends to be poor due to a deficiency in the sulfur-containing amino acids, methionine and cysteine. Suboptimal levels of essential amino acids can lead to protein-energy malnutrition (PEM), which is characterized by increased susceptibility to disease, decreased levels of blood proteins, and impaired mental and physical development in children. It is estimated by the World Health Organization that 30% of the population in developing countries suffer from PEM (Onis et al., Bull World Health Organ., 71: 703-712, 1993). Among the essential amino acids, methionine, lysine, and tryptophan are of particular interest, as lysine and tryptophan are the most limiting amino acids in cereals, while methionine is most limiting in legumes.

### SUMMARY

Increasing the amount of limiting amino acids (e.g., methionine, lysine, and tryptophan, and/or cysteine) in plants such as legumes and cereal grains may result in enhanced value for producers and consumers. The materials and methods described herein can be used to generate plants having amino acid profiles with increased amounts of limiting amino acids, particularly through decreasing the levels of proteins with undesired amino acid content.

This document is based, at least in part, on the discovery plant soybean varieties having altered content of one or more particular amino acids can be obtained by using sequence-specific nucleases to cleave DNA sequences within or near loci encoding particular polypeptides. For example, this document is based, at least in part, on the discovery that soybean varieties having increased sulfur-containing amino acid content can be obtained by using sequence-specific nucleases to cleave DNA sequences within or near loci containing coding sequences for glycinin and/or conglycinin, which are the major seed storage proteins in soybean. Thus, this document provides methods for using sequence-specific nucleases to generate soybean varieties with reduced copy numbers of functional low level sulfur-containing globulin genes, reduced expression of low level sulfur-containing globulin genes, and/or reduced levels of low level sulfur-containing globulin proteins, including Gy4 and Gy5 glycinin, and β-subunit conglycinin. For example, delivery of sequence-specific nucleases can result in targeted knockout or targeted deletion of low sulfur-containing glycinin or conglycinin sequences, and subsequently can result in decreased levels of (a) mRNA encoding low sulfur-containing glycinin/conglycinin, and (b) low sulfur-containing glycinin/conglycinin protein within soybean seeds. The seeds from the modified soybean varieties provided herein, as compared to seeds from non-modified soybean, can have reduced content of low-level sulfur-containing globulin proteins and, as a result of rebalancing, may have increased levels of high sulfur-containing proteins. Such seeds may be useful as a healthier protein source for human and animal consumption.

This document is also based, at least in part, on the development of soybean varieties with mutations within or near glycinin and conglycinin genes that are created using sequence-specific nucleases. The resulting improved sulfur-containing globulin levels in these soybean varieties can be achieved without insertion of a transgene. There are several challenges for commercializing transgenic plants, including strict regulation in certain jurisdictions, which can result in high costs to obtain regulatory approval. The methods described herein can accelerate the production of new soybean varieties with improved sulfur-containing globulin content, and can be more cost-effective than transgenic or traditional breeding approaches.

In a first aspect, this document features a plant, plant part, or plant cell having a mutation in at least one seed storage protein gene that is endogenous to the plant, plant part, or plant cell, wherein the plant, plant part, or plant cell has altered amino acid content as compared to a control plant, plant part or plant cell that lacks the mutation. The mutation can have been introduced using a rare-cutting endonuclease [e.g., a transcription activator-like effector (TALE) nuclease, meganuclease, zinc finger nuclease (ZFN), or clustered regularly interspaced short palindromic repeat (CRISPR)/Cas reagent]. The at least one seed storage protein gene can be selected from the group consisting of a glycinin gene, a beta-conglycinin gene, a glutenin gene, a gliadin gene, a zein gene, a hordein gene, a secalin gene, and a prolamine gene. The mutation can be a deletion of one or more base pairs. The deletion can be at a target sequence as set forth in SEQ ID NO:1 or SEQ ID NO:2, or at a target sequence with at least 90% identity to the sequence set forth in SEQ ID NO:1 or SEQ ID NO:2. The deletion can be at a target sequence as set forth in SEQ ID NO:17 or SEQ ID NO:18, or at a target sequence with at least 90% identity to SEQ ID NO:17 or SEQ ID NO:18. The deletion can be at a target sequence as set forth in SEQ ID NO:9, SEQ ID NO:10, or SEQ ID NO:11, or at a target sequence with at least 90% identity to SEQ ID NO:9, SEQ ID NO:10, or SEQ ID NO:11. In some cases, the at least one seed storage protein gene can include a Gy4 gene, a Gy5 gene, or a beta-conglycinin gene. The mutation can be a deletion of one or more base pairs within a Gy4 gene that results in a sequence as set forth in any of SEQ ID NOS:6390-6396 and 6408-6422, or the mutation can be a deletion within a Gy5 gene that results in a sequence as set forth in any of SEQ ID NOS:6353-6366, 6379-6388, 6397-6400, and 6404-6406. The altered amino acid content can include an increase in methionine or cysteine content as compared to a corresponding control plant, plant part, or plant cell that lacks the mutation. In some cases, the at least one seed storage protein gene can include an alpha-gliadin gene, an omega-gliadin gene, or a gamma-gliadin gene. The mutation can be a deletion of one or more base pairs. The deletion can be at a target sequence as set forth in any of SEQ ID NOS:6367-6370, or at a target sequence with at least 90% identity to any of SEQ ID NOS:6367-6370. The altered amino acid content can include an increase in lysine content as compared to a corresponding control plant, plant part, or plant cell that lacks the mutation.

In another aspect, this document features a method for making a plant having altered amino acid content. The method can include (a) contacting plant cells or plant parts having functional seed storage protein genes with a rare-cutting endonuclease targeted to a sequence within one or more of the functional seed storage protein genes, or to a sequence flanking the functional seed storage protein genes; (b) growing the contacted plant cells or plant parts into plants; and (c) selecting, from the plants, a plant with a mutation in at least one seed storage protein gene. The rare-cutting endonuclease can be a TALE nuclease, meganuclease, ZFN, or CRISPR/Cas reagent. The at least one seed storage protein gene can be selected from the group consisting of a glycinin gene, a beta-conglycinin gene, a glutenin gene, a gliadin gene, a zein gene, a hordein gene, a secalin gene, and a prolamine gene. The mutation can be a deletion of one or more base pairs. The deletion can be at a target sequence as set forth in SEQ ID NO:1 or SEQ ID NO:2, or at a target sequence with at least 90% identity to the sequence set forth in SEQ ID NO:1 or SEQ ID NO:2. The deletion can be at a target sequence as set forth in SEQ ID NO:17 or SEQ ID NO:18, or at a target sequence with at least 90% identity to SEQ ID NO:17 or SEQ ID NO:18. The deletion can be at a target sequence as set forth in SEQ ID NO:9, SEQ ID NO:10, or SEQ ID NO:11, or at a target sequence with at least 90% identity to SEQ ID NO:9, SEQ ID NO:10, or SEQ ID NO:11. In some cases, the at least one seed storage protein gene can include a Gy4 gene, a Gy5 gene, or a beta-conglycinin gene. The mutation can be a deletion of one or more base pairs within a Gy4 gene that results in a sequence as set forth in any of SEQ ID NOS:6390-6396 and 6408-6422, or the mutation can be a deletion within a Gy5 gene that results in a sequence as set forth in any of SEQ ID NOS:6353-6366, 6379-6388, 6397-6400, and 6404-6406. The altered amino acid content can include an increase in methionine or cysteine content as compared to a corresponding control plant that lacks the mutation. In some cases, the at least one seed storage protein gene can include an alpha-gliadin gene, an omega-gliadin gene, or a gamma-gliadin gene. The mutation can be a deletion of one or more base pairs. The deletion can be at a target sequence as set forth in any of SEQ ID NOS:6367-6370, or at a target sequence with at least 90% identity to any of SEQ ID NOS:6367-6370. The altered amino acid content can include an increase in lysine content as compared to a corresponding control plant, plant part, or plant cell that lacks the mutation.

In another aspect, this document features a method for mutagenizing a cell. The method can include (a) treating the cell with an agent (e.g., a chemical) that reduces DNA methylation or interferes with histone deacetylase activity; and (b) contacting the cell with a rare-cutting endonuclease. The cell can be a plant cell. The agent can be 5-azacytidine or trichostatin A. The rare-cutting endonuclease can be a TALE nuclease, meganuclease, ZFN, or CRISPR/Cas reagent.

In another one aspect, this document features a plant, plant part, or plant cell having a mutation in at least one seed storage protein gene that is endogenous to the plant, plant part, or plant cell, where the plant, plant part, or plant cell has reduced content of the seed storage protein as compared to a control plant, plant part or plant cell that lacks the mutation. In some cases, the plant, plant part, or plant cell can be a soybean plant, plant part or plant cell. The seed storage protein gene can be selected from the group consisting of a Gy4 gene, a Gy5 gene, and a beta-conglycinin gene. The mutation can be at a target sequence as set forth in SEQ ID NO:1, SEQ ID NO:2, SEQ ID NO:3, or SEQ ID NO:4, or at a target sequence that, when translated, has at least 90 percent amino acid identity to the sequence set forth in SEQ ID NO:6, SEQ ID NO:7, SEQ ID NO:8, or SEQ ID NO:9. The mutation can have been introduced using a rare-cutting endonuclease (e.g., a transcription activator-like effector (TALE) nuclease, meganuclease, zinc finger nuclease (ZFN), or clustered regularly interspaced short palindromic repeat (CRISPR)/Cas reagent). The plant, plant part, or plant cell can have a sulfur-containing amino acid content that is at least 0.01% greater than a corresponding plant, plant part, or plant cell that lacks the mutation. The plant, plant part, or plant cell can be a Glycine max L. Merr. plant, plant part, or plant cell. In some cases, the plant, plant part, or plant cell can be a wheat plant, plant part or plant cell. The seed storage protein gene can be selected from the group consisting of an alpha-gliadin gene, and omega-gliadin gene, and a gamma-gliadin gene. The mutation can have been introduced using a rare-cutting endonuclease (e.g., a TALE nuclease, meganuclease, ZFN, or CRISPR/Cas reagent).

In another aspect, this document features a method for making a plant having a targeted mutation in at least one seed storage protein gene. The method can include (a) contacting plant cells or plant parts containing functional seed storage protein genes with a rare-cutting endonuclease targeted to a sequence within one or more of the functional seed storage protein genes, or to a sequence flanking the functional seed storage protein genes, (b) selecting from the plant cells or plant parts of step (a) a plant cell or plant part in which at least one functional seed storage protein gene has been inactivated, and (c) growing the selected plant cell or plant part into a plant, where the plant has reduced levels of the seed storage protein as compared to a control plant in which the seed storage protein gene was not inactivated. The plant cells or plant parts contacted in step (a) can be selected from the group consisting of immature embryos, leaf base explants, hypocotyl explants, embryogenic calli, embryos, scutella, embryonic cell suspension, callus, meristems, microspores, pollen, leaf tissue, seeds, protoplasts, and internode explants. In some cases, the plant, plant part, or plant cell can be a soybean plant, plant part or plant cell. The seed storage protein gene can be selected from the group consisting of a Gy4 gene, a Gy5 gene, and a beta-conglycinin gene. The mutation can be at a target sequence as set forth in SEQ ID NO:1, SEQ ID NO:2, SEQ ID NO:3, or SEQ ID NO:4, or at a target sequence that, when translated, has at least 90 percent amino acid identity to the sequence set forth in SEQ ID NO:6, SEQ ID NO:7, SEQ ID NO:8, or SEQ ID NO:9. The mutation can have been introduced using a rare-cutting endonuclease (e.g., a TALE nuclease, meganuclease, ZFN, or CRISPR/Cas reagent). The selected soybean plant, plant part, or plant cell can have a sulfur-containing amino acid content that is at least 0.01% greater than the sulfur-containing amino acid content of a corresponding soybean plant, plant part, or plant cell that lacks the mutation. The soybean plant, plant part, or plant cell can be a Glycine max L. Merr. plant, plant part, or plant cell. In some cases, the plant, plant part, or plant cell can be a wheat plant, plant part or plant cell. The seed storage protein gene can be selected from the group consisting of an alpha-gliadin gene, an omega-gliadin gene, and a gamma-gliadin gene. The mutation can have been introduced using a rare-cutting endonuclease (e.g., a TALE nuclease, meganuclease, ZFN, or CRISPR/Cas reagent).

In another aspect, this document features a soybean plant, plant part, or plant cell having a targeted mutation in at least one low sulfur-containing globulin gene that is endogenous to the plant, plant part, or plant cell, wherein the plant, plant part, or plant cell has reduced low sulfur-containing globulin content as compared to a control soybean plant, plant part, or plant cell that lacks the mutation. The mutation can be a deletion of one or more nucleotide base pairs, a substitution of one or more nucleotide base pairs, or an insertion of one or more nucleotide base pairs. The mutation can be a deletion of one or more low sulfur-containing globulin genes. The mutation can include a combination of two or more of: deletion of one or more genes, inversion of one or more genes, insertion of one or more nucleotides within a gene, deletion of one or more nucleotides from a gene, and substitution of one or more nucleotides within a gene. The mutation can be at a target sequence as set forth in SEQ ID NO:1, SEQ ID NO:2, SEQ ID NO:3, or SEQ ID NO:4, or at a target sequence that, when translated, has at least 90 percent amino acid identity to an amino acid sequence encoded by SEQ ID NO:1, SEQ ID NO:2, SEQ ID NO:3, or SEQ ID NO:4. The low sulfur-containing globulin content can include globulin DNA, globulin mRNA, and/or globulin protein. The plant, plant part, or plant cell can have been made using a rare-cutting endonuclease (e.g., a transcription activator-like effector (TALE) endonuclease, also referred to herein as a TALE nuclease). The TALE nuclease can bind to a sequence as set forth in any of SEQ ID NO:1, SEQ ID NO:2, SEQ ID NO:3, or SEQ ID NO:4, or binds to a sequence that, when translated, has at least 90 percent amino acid identity to an amino acid sequence encoded by SEQ ID NO:1, SEQ ID NO:2, SEQ ID NO:3, or SEQ ID NO:4. The TALE nuclease can bind to a sequence that flanks a sequence as set forth in SEQ ID NO:1, SEQ ID NO:2, SEQ ID NO:3, or SEQ ID NO:4, or that flanks a sequence that, when translated, has at least 90 percent amino acid identity to an amino acid sequence encoded by SEQ ID NO:1, SEQ ID NO:2, SEQ ID NO:3, or SEQ ID NO:4. Each of the one or more low sulfur-containing globulin genes having a mutation can exhibit deletion, substitution, or insertion of an endogenous nucleic acid, without including any exogenous nucleic acid. In some embodiments, two or more endogenous low sulfur-containing globulin genes can contain a mutation. The plant, plant part, or plant cell can have a sulfur-containing amino acid content that is at least 0.01% greater than a corresponding soybean plant, plant part, or plant cell that lacks the mutation. The plant, plant part, or plant cell is a Glycine max L. Men. plant, plant part, or plant cell.

In another aspect, this document features a method for making a soybean plant having reduced low sulfur-containing globulin content. The method can include (a) contacting soybean plant cells or plant parts having functional globulin genes with a rare-cutting endonuclease targeted to sequence within one or more of the functional globulin genes, or to sequence flanking the globulin genes, (b) selecting from the plant cells or plant parts a plant cell or plant part in which at least one globulin gene has been inactivated, and (c) growing the selected plant cell or plant part into a soybean plant, wherein the soybean plant has reduced low sulfur-containing globulin content as compared to a control soybean plant in which the globulin gene has not been inactivated. The soybean plant cells contacted in step (a) can be protoplasts. The method can include transforming the protoplasts with a nucleic acid encoding the rare-cutting endonuclease. The nucleic acid can be an mRNA. The nucleic acid can be contained within a vector. The soybean plant parts contacted in step (a) can be immature embryos or embryogenic calli. The method can include transformation of the embryos or embryogenic calli with a nucleic acid encoding the rare-cutting endonuclease. The transformation can be Agrobacterium-mediated transformation or transformation by biolistics. The rare-cutting endonuclease can be a TALE nuclease, meganuclease, ZFN, or CRISPR/Cas reagent. The method can further include culturing the protoplasts, immature embryos, or embryogenic calli to generate plant lines. Each mutation can be at a target sequence as set forth in SEQ ID NO:1, SEQ ID NO:2, SEQ ID NO:3, or SEQ ID NO:4, or at a target sequence that, when translated, has at least 90 percent amino acid identity to an amino acid sequence encoded by SEQ ID NO:1, SEQ ID NO:2, SEQ ID NO:3, or SEQ ID NO:4. The rare-cutting endonuclease can be a TALE nuclease (e.g., a TALE nuclease that binds to sequence that flanks sequence as set forth in SEQ ID NO:1, SEQ ID NO:2, SEQ ID NO:3, or SEQ ID NO:4, or that flanks a sequence that, when translated, has at least 90 percent amino acid identity to an amino acid sequence encoded by SEQ ID NO:1, SEQ ID NO:2, SEQ ID NO:3, or SEQ ID NO:4). In some embodiments, two or more functional endogenous globulin genes can be mutated. The soybean plant can have a sulfur-containing amino acid level of at least 3%. The soybean plant, plant part, or plant cell can be a Glycine max L. Men. plant, plant part, or plant cell. The method can include isolating genomic DNA containing at least a portion of the globulin gene from the protoplasts, immature embryos, or embryogenic calli.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1C show representative Gy4 glycinin Glyma10g04280 sequences. FIG. 1A is an example of a Gy4 glycinin Glyma10g04280 coding sequence (SEQ ID NO:1) that can be a target for TALE nuclease-mediated gene inactivation. FIG. 1B is an example of a Gy4 glycinin Glyma10g04280 genomic sequence (SEQ ID NO:16) that can be a target for TALE nuclease-mediated gene inactivation. Underlined nucleotides indicate 5′ and 3′ UTR sequences. Lower case nucleotides indicate intronic sequences. FIG. 1C is a fragment of the Gy4 glycinin Glyma10g04280 genomic sequence (SEQ ID NO:17) that can be a target for TALE nuclease-mediated gene inactivation.

FIGS. 2A-2C show representative Gy5 glycinin Gyma13g18450 sequences. FIG. 2A is an example of a Gy5 glycinin Glyma13g18450 coding sequence (SEQ ID NO:2) that can be a target for TALE nuclease-mediated gene inactivation. FIG. 2B is an example of a Gy5 glycinin Glyma13g18450 genomic sequence (SEQ ID NO:18) that can be a target for TALE nuclease-mediated gene inactivation. Lower case nucleotides indicate intronic sequences. FIG. 2C is a fragment of the Gy5 glycinin Glyma13g18450 genomic sequence (SEQ ID NO:19) that can be a target for TALE nuclease-mediated gene inactivation.

FIG. 3 is an example of a beta-conglycinin Glyma20g28460 coding sequence (SEQ ID NO:3) that can be a target for TALE nuclease-mediated gene inactivation.

FIG. 4 is an example of a beta-conglycinin Glyma20g28640 coding sequence (SEQ ID NO:4) that can be a target for TALE nuclease-mediated gene inactivation.

FIG. 5 is an example of a Gy4 glycinin Glyma10g04280 amino acid sequence (SEQ ID NO:5) that can be targeted by TALE nuclease-mediated gene inactivation. Capital letters indicate sulfur-containing amino acids.

FIG. 6 is an example of a Gy5 glycinin Glyma13g18450 amino acid sequence (SEQ ID NO:6) that can be targeted by TALE nuclease-mediated gene inactivation. Capital letters indicate sulfur-containing amino acids.

FIG. 7 is an example of a beta-conglycinin Glyma20g28460 amino acid sequence (SEQ ID NO:7) that can be targeted by TALE nuclease-mediated gene inactivation. Capital letters indicate sulfur-containing amino acids.

FIG. 8 is an example of a beta-conglycinin Glyma20g28640 amino acid sequence (SEQ ID NO:8) that can be targeted by TALE nuclease-mediated gene inactivation. Capital letters indicate sulfur-containing amino acids.

FIG. 9 lists examples of TALE nuclease targeting sequences (SEQ ID NOS:9-14) that can be used for inactivating low sulfur-containing globulin genes. Bold font indicates half TALE nuclease targeting sequences; underlining indicates spacer sequences.

FIGS. 10A and 10B are exemplary illustrations of the methods described herein for altering amino acid composition in plants. FIG. 10A shows a hypothetical “normal” condition within a plant cell, where Expressed Gene 1 produces Protein 1 at large quantities and Compensation Gene 2 produces Protein 2 at low levels. The amino acid composition of both proteins is shown. The low frequency of the amino acids M (methionine) and C (cysteine) within Protein 1 contributes to the low frequency of M and C in the plant part (right graph). The high frequency of H (histidine) in Protein 1 contributes to the high frequency of H in the plant part. FIG. 10B demonstrates a hypothetical situation in which Expressed Gene 1 is knocked out or has reduced expression, and Compensation Gene 2 compensates for Expressed Gene 1 and Protein 1. The high frequency of M and C in Protein 2 contributes to a higher frequency of M and C in the plant part.

FIG. 11 is an example of an amino acid sequence for an alpha-gliadin protein from wheat (T. aestivum; SEQ ID NO:20).

FIG. 12 is an example of an amino acid sequence for a gamma-gliadin protein from wheat (T. aestivum; SEQ ID NO:21).

FIG. 13 is an example of an amino acid sequence for an omega-gliadin protein from wheat (T. aestivum; SEQ ID NO:22).

FIG. 14 shows the nucleotide target sequence of TaGliadin TALE nuclease pairs (SEQ ID NOS: 6367-6370). Bold font indicates half TALE nuclease target sequences; underlining indicates spacer sequences.

FIG. 15 shows nuclease-induced deletions in the alpha-gliadin genes (SEQ ID NOS:6367 and 6371-6378).

FIGS. 16A and 16B show nuclease-induced deletions in the soybean Gy5 gene (FIG. 16A; SEQ ID NOS:6379-6388) and Gy4 gene (FIG. 16B; SEQ ID NOS:6389-6396).

FIG. 17 shows nuclease induced mutations in the Gy4 and Gy5 genes in a T2 plant that is progeny of the T1 parent plant Gm318-1-4.

FIG. 18 shows nuclease induced mutations in the Gy4 and Gy5 genes in a T2 plant (plant 1) that is progeny of the T1 parent plant Gm318-1-2.

FIG. 19 shows nuclease induced mutations in the Gy4 and Gy5 genes in a T2 plant (plant 2) that is progeny of the T1 parent plant Gm318-1-2.

FIG. 20 shows nuclease induced mutations in the Gy4 and Gy5 genes in a T2 plant (plant 3) that is progeny of the T1 parent plant Gm318-1-2.

### DETAILED DESCRIPTION

This document is based, at least in part, on the discovery that content of individual amino acids within plants, plant cells, or plant parts can be altered (e.g., increased or decreased) through the use of one or more sequence-specific nucleases to cleave DNA sequences within or near loci encoding particular proteins that are expressed in the plants, plant cells, or plant parts. The cleavage may result in downregulation or complete loss of certain protein expression in the plants, plant cells, or plant parts. The cleavage may result in inactivation or knockout of the protein. The downregulation, complete loss of expression, or inactivation of a certain protein can trigger a compensation mechanism that may result in increased expression of one or more other proteins (referred to herein as “compensation proteins”) that were not targeted by the sequence-specific nuclease(s). Compensation proteins can have a different amino acid content than the protein with reduced or lost expression. The downregulation, complete loss of expression, or inactivation of a certain protein, together with increased expression of one or more compensation proteins, can result in altered amino acid content in the plants, plant cells, or plant parts. Target proteins for downregulation or inactivation typically harbor one or more amino-acids-of-interest at a percent-total of the amino acids within the protein that is less than the overall percent-total of the amino-acids-of-interest within all proteins combined in the plant, plant part, or plant cell.

Thus, this document is based, at least in part, on the discovery that downregulation, complete loss of expression, or inactivation of certain proteins can result in increased content of particular amino acids, relative to the total amino acid content, in plants, plant cells, or plant parts, and also can result in decreased content of particular amino acids, relative to the total amino acid content, in the plants, plant cells, or plant parts. Downregulation, complete loss of expression, or inactivation of a certain protein can be achieved using one or more (e.g., one, two three, four, five, six, or more than six) sequence-specific nucleases. For example, inactivation of a protein can be achieved by introducing one or more mutations (e.g., nucleotide substitutions, deletions, or insertions) within the nucleic acid sequence of the gene encoding the protein (e.g., within the coding sequence). The one or more mutations can, in some cases, be a deletion that results in a frameshift that may lead to an early stop codon and potentially nonsense mediated decay (if the early stop codon occurs before an intron). If a frameshift mutation occurs near the end of the coding sequence and after the last intron, then majority of the protein may still be produced. If a frameshift mutation occurs near the beginning of the coding sequence, then the majority of the protein will not likely be produced. Thus, in some cases, frameshift mutations occurring at or near the beginning of a coding sequence can be particularly useful.

In some embodiments, an insertion or deletion of nucleotides (nt) within a gene can have a length of about 1 nt to about 10,000 nt (e.g., 1 to 10 nt, 5 to 15 nt, 10 to 25 nt, 20 to 50 nt, 50 to 100 nt, 100 to 200 nt, 200 to 500 nt, 500 to 1000 nt, 1000 to 2000 nt, 2000 to 3000 nt, 3000 to 4000 nt, 4000 to 5000 nt, or 5000 to 10,000 nt). In some cases, when the mutation is a deletion, at least about 0.05% (e.g., at least about 0.1%, at least about 0.15%, at least about 0.2%, at least about 0.25%, at least about 0.3%, at least about 0.5%, at least about 1%, at least about 2%, about 0.05 to 0.1%, about 0.1 to 0.15%, about 0.15 to 0.2%, about 0.2 to 0.25%, about 0.25 to 0.3%, about 0.3 to 0.4%, about 0.4 to 0.5%, about 0.5 to 0.75%, about 0.75 to 1%, about 1 to 2%, or about 2 to 3%) of the nucleotides within a gene can be deleted.

As used herein, the term “amino acid content” with respect to a particular amino acid refers to the percentage of that particular amino acid among the total amount of amino acids within a population (e.g., in a protein, a plant, a plant part, or a plant cell). When referring to a plant, plant part, or plant cell, “amino acid content” refers to the percentage of a certain amino acid among the total amount of amino acids within the plant, plant part, or plant cell. When referring to a protein, “amino acid content” refers to the percentage of a certain amino acid among the total amino acids within the protein.

The plant, plant part, can plant cells provided herein can have a mutation that results in an altered amino acid content, such that the amount of one or more amino acids is at least about 0.01% (e.g., at least about 0.02%, at least about 0.05%, at least about 0.1%, at least about 0.5%, at least about 1%, at least about 3%, at least about 5%, about 0.01 to 0.1%, about 0.05 to 0.5%, about 0.1 to 1%, about 0.2 to 1.5%, about 0.5 to 2%, about 1 to 3%, or about 2 to 5%) greater or less than the amount of that amino acid in a corresponding plant, plant part, or plant cell that lacks the mutation. For example, if a plant, plant part, or plant cell that lacks the mutation has a content of a particular amino acid that is about 5.00% of the total amino acids, and the mutation results in an increase in content of the particular amino acid, then the plant, plant part, or plant cell that contains the mutation can have a content of the particular amino acid of at least 5.01% (e.g., at least about 5.02%, at least about 5.05%, at least about 5.10%, at least about 5.50%, at least about 6.00%, at least about 8.00%, at least about 10.00%, about 5.01 to 5.10%, about 5.05 to 5.50%, about 5.50 to 6.00%, about 5.20 to 6.50%, about 5.50 to 8.00%, about 6.00 to 8.00%, or about 7.00 to 10.00%). Methods for generating such plant varieties also are provided herein.

Thus, in some embodiments, this document provides methods for making plants having altered amino acid content. The methods can include, for example, contacting plant cells or plant parts having functional seed storage protein genes with a sequence-specific, rare-cutting endonuclease targeted to a sequence within one or more of the functional seed storage protein genes, growing the contacted plant cells or plant parts into plants, and selecting a plant with a mutation in at least one seed storage protein gene. In some cases, the heterochromatic state of particular genes may hinder or prevent an endonuclease from binding and cleaving DNA. In such cases, an agent that reduces DNA methylation or reduces histone deacetylase activity can be used to relax the chromatin and allow access to the target sequences. Thus, the methods provided herein may include the step of treating a cell (e.g., a plant cell or a mammalian cell) or a plant part with an agent (e.g., 5-azacytidine or trichostatin A) that reduces DNA methylation or interferes with histone deacetylase activity, and then contacting the cell or plant part with the sequence-specific, rare-cutting endonuclease.

In some embodiments, one or more sequence-specific nucleases can be used to achieve downregulation, complete loss of expression, or inactivation of one or more proteins within a cereal plant. The one or more proteins can be, without limitation, seed storage proteins, which include prolamines, albumins, and globulins. In some cases, the cereal that can be modified with the methods described herein can be within the family Poaceae. In some cases, the cereal can be, without limitation, rice, bread wheat (Triticum aestivum), durum wheat (Triticum durum), corn, barley, millet, sorghum, rye, triticale, teff, wild rice, spelt, buckwheat, or quinoa.

In some embodiments, one or more sequence-specific nucleases can be used to achieve downregulation, complete loss of expression, or inactivation of one or more proteins within a legume. The one or more proteins can be, for example, seed storage proteins. In some cases, the legume that can be modified with the methods described herein can be within the family Fabaceae. In some cases, the legume can be, without limitation, soybean, asparagus, green bean, kidney bean, navy bean, pinto bean, garbanzo bean, adzuki bean, Anasazi bean, wax bean, mung bean, dwarf pea, southern pea, English pea, snow pea, sugar snap pea, alfalfa, clover, lentils, or peanut.

Although soybean has the highest protein content among seed crops, the protein quality is poor due to a deficiency in the sulfur-containing amino acids, methionine and cysteine. This document therefore provides soybean plant varieties, particularly those of the species Glycine max L. Merr., which contain reduced (or even no) detectable levels of low sulfur-containing globulin proteins, and have increased levels of sulfur-containing amino acids. In some embodiments, for example, a soybean plant, plant part, or plant cell as provided herein can have a mutation that results in a sulfur-containing amino acid content that is at least about 0.01% (e.g., at least about 0.02%, at least about 0.05%, at least about 0.1%, at least about 0.5%, at least about 1%, at least about 3%, at least about 5%, about 0.01 to 0.1%, about 0.05 to 0.5%, about 0.1 to 1%, about 0.2 to 1.5%, about 0.5 to 2%, about 1 to 3%, or about 2 to 5%) greater than the sulfur-containing amino acid content of a corresponding soybean plant, plant part, or plant cell that lacks the mutation. For example, if a soybean plant, plant part, or plant cell that lacks the mutation has a sulfur-containing amino acid content of 1.61%, then the soybean plant, plant part, or plant cell that contains the mutation can have a sulfur-containing amino acid content of at least about 1.62% (e.g., at least about 1.63%, at least about 1.66%, at least about 1.71%, at least about 2.11%, at least about 2.61%, at least about 4.61%, at least about 6.61%, about 1.62 to 1.71%, about 1.66 to 2.11%, about 1.71 to 2.61%, about 1.81 to 3.11%, about 2.11 to 4.61%, about 2.61 to 4.61%, or about 3.61 to 6.61%). Methods for generating such soybean plant varieties also are provided herein.

Soybean 7S globulin (β-conglycinin) and 11S globulin (glycinin) are the two major protein components of the seed, accounting for about 70% of the total seed protein at maturity, and about 30%-40% of the mature seed weight. Other major proteins in soybean seeds include urease, lectin, and trypsin inhibitors. The 11S and 7S soybean seed storage proteins usually are identified by their sedimentation rates in sucrose gradients (Hill and Breidenbach, Plant Physiol, 53:747-751, 1974). The content of sulfur-containing amino acids in the two globulins is very different; 11S globulin contains three to four times more methionine and cysteine per unit protein than 7S globulin.

The 11S protein (glycinin, legumin) contains at least four acidic subunits and four basic subunits (Staswick et al., J Biol Chem, 256:8752-8755, 1981), which form combined subunits designated A1B1, A1B2, A2B1, A3B4, and A4A5B3. The acidic and basic subunits are produced by cleavage of precursor polypeptides, which originally were identified through in vitro translation and pulse-labeling experiments (Barton et al., J Biol Chem, 257:6089-6095, 1982). The 7S storage protein (conglycinin, vicilin) is a glycoprotein composed of three major subunits, designated the α, α′ and β-subunits (Beachy et al., J Mol Appl Genet, 1:19-27, 1981).

Each subunit of 115 and 7S varies in the content of sulfur-containing amino acids. 11S glycinin is encoded by the Gy1 through Gy8 genes. Gy1-Gy5 are highly expressed in developing soybean seeds, while Gy7 expressed at low levels, and Gy6 and Gy8 are pseudogenes. Of the 7S β-conglycinin genes, Glyma10g39150 encodes the α′-subunit, Glyma20g28650 and Glyma20g28660 encodes the α-subunit, and Glyma20g28460 and Glyma20g28640 encodes the β-subunit.

In some embodiments, the plant can be a soybean plant and the one or more target genes for downregulation or inactivation can be the beta-conglycinin (7S) and/or glycinin (11S) seed storage protein genes. Since beta-conglycinin and glycinin are naturally low in methionine and cysteine, knockout or knockdown of one or more beta-conglycinin or glycinin genes can result in compensation of other proteins with higher levels of methionine and cysteine. Thus, knockout or knockdown of one or more beta-conglycinin or glycinin genes can result in an overall increase in the levels of methionine and cysteine in the soybean seed. Additional details about soybean seed storage proteins, including their structure and function, can be found elsewhere (see, e.g., Li et al., Heredity, 106:633-641, 2011; and Shewry et al., The Plant Cell, 7:945-956, 1995).

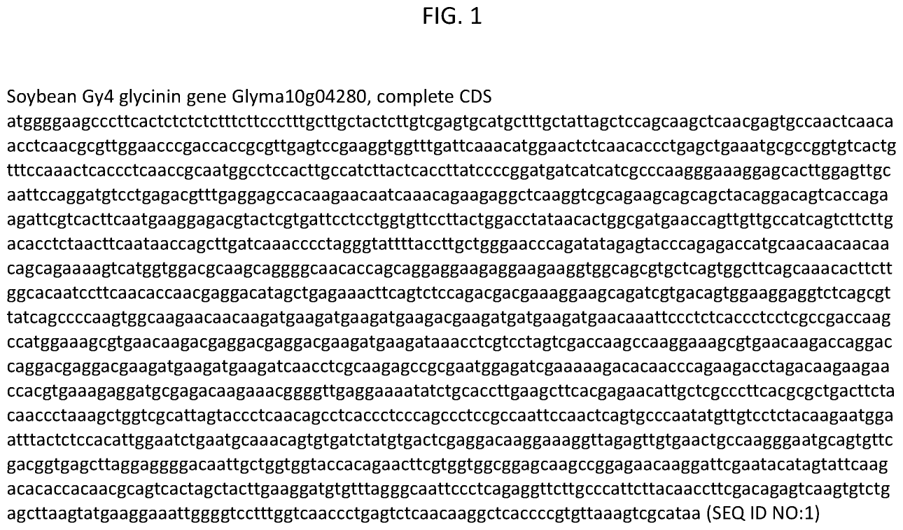

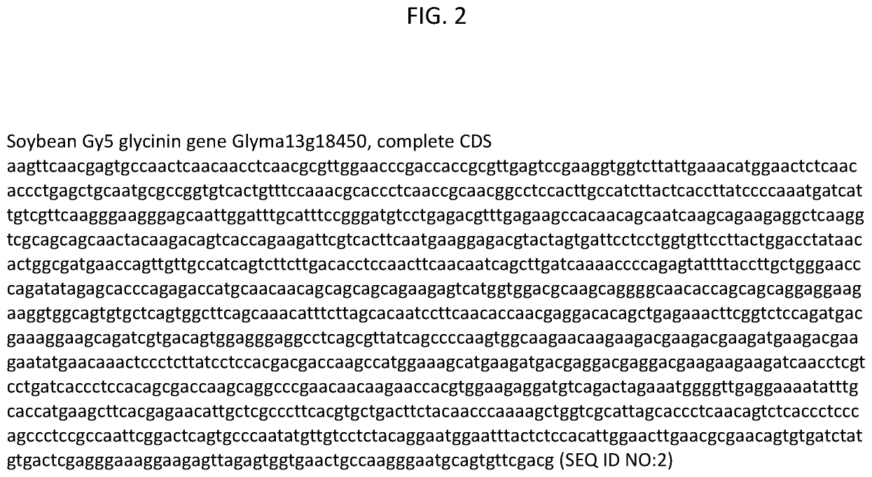

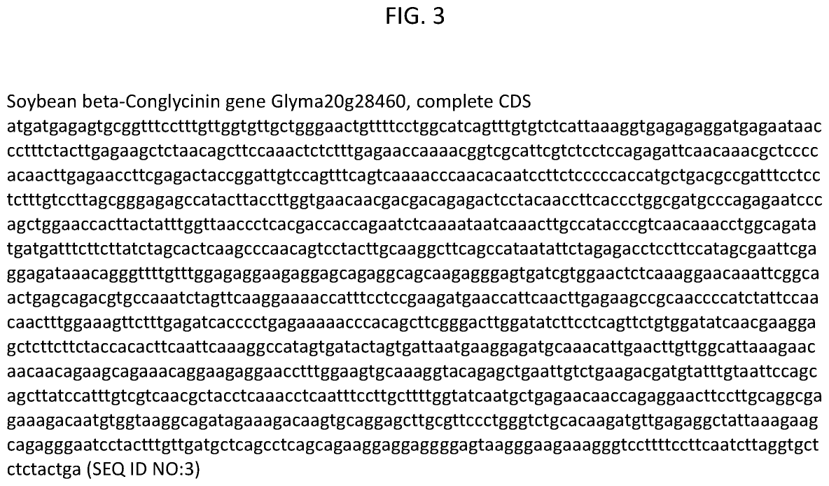

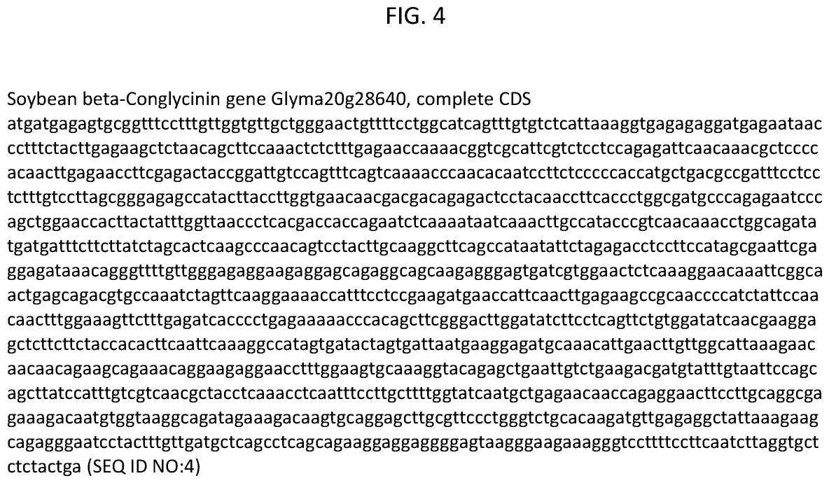

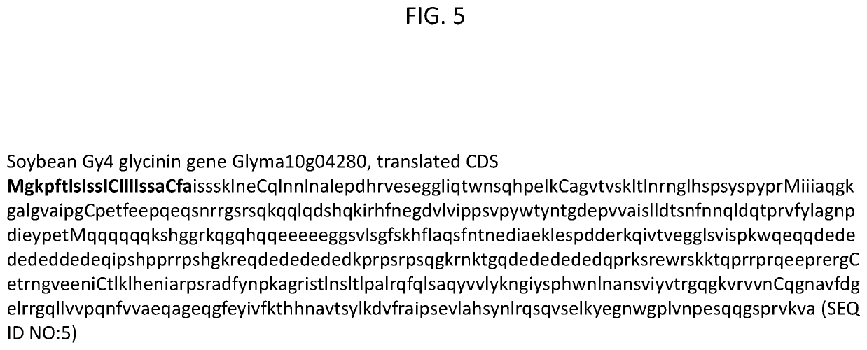

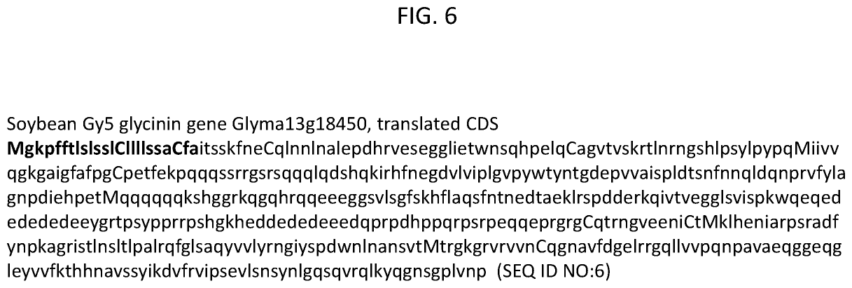

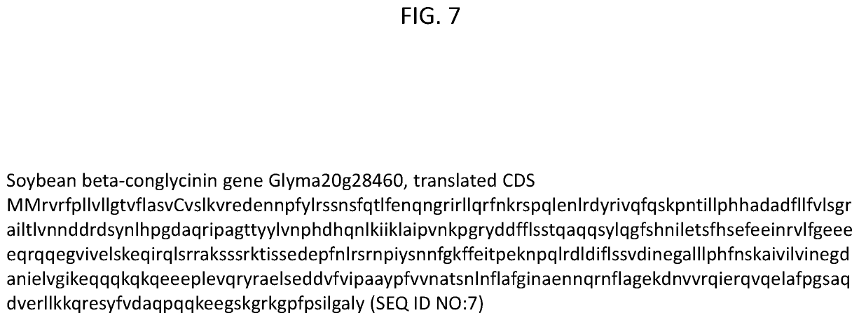

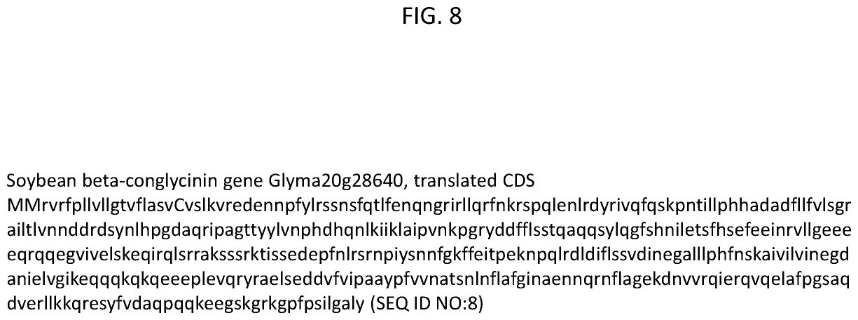

Examples of glycinin genes that can be downregulated or inactivated include Gy1 (A1B2; Glyma03g32030), Gy2 (A2B1; Glyma03g32020), Gy3 (A1B1; Glyma19g34780), Gy4 (A5A4B3; Glyma10g04280, with representative sequences set forth as SEQ ID NOS:1, 16, and 17 in FIGS. 1A, 1B, and 1C, respectively), and Gy5 (A3B4; Glyma13g18450, with representative sequences set forth as SEQ ID NOS: 2, 18, and 19 in FIGS. 2A, 2B, and 2C, respectively). Examples of beta-conglycinin genes that can be downregulated or inactivated include Glyma20g28460 (SEQ ID NO:3, FIG. 3) and Glyma20g28640 (SEQ ID NO:4, FIG. 4). An example of a Gy4 glycinin Glyma10g04280 amino acid sequence that can be targeted for gene inactivation is shown in FIG. 5 (SEQ ID NO:5). An example of a Gy5 glycinin Glyma13g18450 amino acid sequence that can be targeted for inactivation is shown in FIG. 6 (SEQ ID NO:6). An example of a beta-conglycinin Glyma20g28460 amino acid sequence that can be targeted for gene inactivation is shown in FIG. 7 (SEQ ID NO:7). An example of a beta-conglycinin Glyma20g28640 amino acid sequence that can be a target for gene inactivation is shown in FIG. 8 (SEQ ID NO:8). Capital letters in FIGS. 5-8 indicate sulfur-containing amino acids.

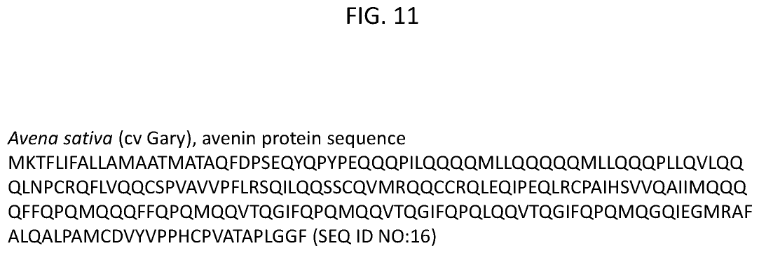

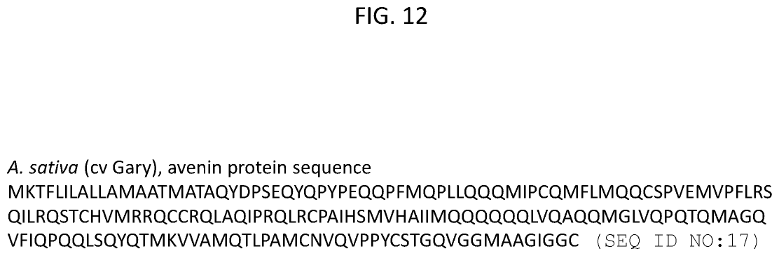

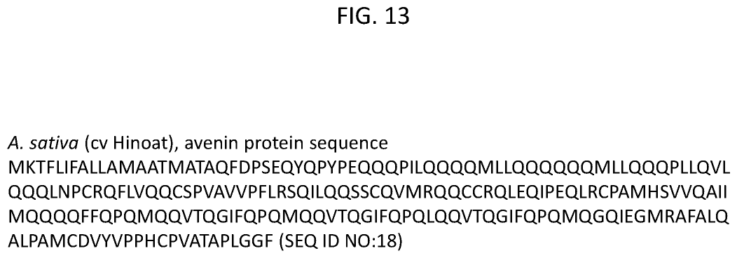

In some embodiments, the plant that can be modified can be a wheat plant, and the one or more target proteins for downregulation or inactivation can be alpha-gliadin, gamma-gliadin, omega-gliadin, and/or glutenin seed storage proteins. Among other amino acids, gliadin proteins are naturally low in lysine. Knocking out or downregulating the expression of gliadin seed storage proteins can result in an overall increase in lysine content in the wheat grain. Examples of alpha-gliadin, gamma-gliadin, and omega-gliadin amino acid sequences for downregulation or inactivation are shown in SEQ ID NOS:20-22 (FIGS. 11-13, respectively). Additional details about the gliadin protein family, including their copy number, structure, and function, can be found elsewhere (see, e.g., Shewry et al., J Exp Bot 53:947-958, 2002; Gil-Humanes et al., Proc Natl Acad Sci USA 107:17023-17028, 2010; and Shewry et al. 1995, supra.

In some embodiments, the plant can be a corn plant, and the one or more target proteins for downregulation or inactivation can be prolamine seed storage proteins (e.g., the alpha-, beta-, gamma-, or delta-zeins; see, Argos et al., J Biol Chem 257:9984-9990, 1982: and Shewry et al. 1995, supra). The zein seed storage proteins are naturally deficient in lysine and tryptophan content. Knocking out or downregulating the expression of zein seed storage protein genes can result in an overall increase in lysine and tryptophan content in the corn seed.

In some embodiments, the plant can be a barley plant and the one or more target proteins for downregulation or inactivation can be hordein seed storage proteins. The hordein seed storage proteins can, for example, be B and gamma-hordeins.

In some embodiments, the plant can be a rye plant and the one or more target proteins for downregulation or inactivation can be secalin seed storage proteins. The secalin seed storage proteins, for example, can be gamma- and omega-secalins.

Plants containing an engineered mutation in a targeted gene also may contain a transgene, which can be integrated into the plant genome using standard transformation protocols (see, for example, Rech et al., Nat Protoc 3:410-418, 2008; Haun et al., Plant Biotech J 12:934-940, 2014; and Curtin et al., Plant Physiol 156:466-473, 2011). The presence and/or expression of the transgene can confer various effects upon the plant. For example, the transgene can result in the expression of a protein that confers tolerance or resistance to an herbicide (e.g., glufonsinate, mesotrione, imidazolinone, isoxaflutole, glyphosate, 2,4-D, hydroxyphenylpyruvate dioxygenase-inhibiting herbicides, or dicamba). The transgene may encode a plant 5-enolpyruvylshikimate-3-phosphate synthase (EPSPS) protein, a bacterial EPSPS protein, an agrobacterium CP4 EPSPS protein, an aryloxyalkanoate dioxygenase (AAD) protein, a phosphinothricin N-acetyltransferase (PAT) protein, a modified acetohydroxyacid synthase large subunit protein, a modified p-hydroxyphenylpyruvate dioxygenase (hppd) protein, or a dicamba monooxygenase (DMO) protein.

In some cases, the transgene can enhance resistance to insects (e.g., lepidopteran insects). For example, the transgene can encode a protein from Bacillus thuringiensis (e.g., a Cry protein, a Cry1Ac delta-endotoxin, a Cry1F delta-endotoxin protein, a Cry2Ab delta-endotoxin protein, or Cry1Ac delta-endotoxin).

The transgene may delay fruit ripening. For example, the transgene can contain an antisense sequence to the polygalacturonase gene.

The transgene can provide enhanced virus resistance. The transgene can contain sequence from a virus genome (e.g., an antisense sequence from a virus genome).

In some cases, the transgene can cause male sterility. For example, the transgene can include a pollen killer gene (e.g., an alpha amylase gene, S24 gene, or S35 gene). The transgene can further contain a screenable marker, such as a fluorescent protein (e.g., GFP, YFP, RFP, or BFP), or a gene involved in regulating seed size. In some cases, the transgene can further contain a restoring factor, such as a functional MS gene (e.g., an MS45 gene).

The transgene may delay browning. For example, the transgene can contain sequence from a polyphenol oxidase gene (e.g., antisense sequence from a polyphenol oxidase gene).

As used herein, the terms “plant” and “plant part” refer to cells, tissues, organs, grains, and severed parts (e.g., roots, leaves, and flowers) that retain the distinguishing characteristics of the parent plant. “Seed” refers to any plant structure that is formed by continued differentiation of the ovule of the plant, following its normal maturation point, irrespective of whether it is formed in the presence or absence of fertilization and irrespective of whether or not the grain structure is fertile or infertile.

The term “allele(s)” means any of one or more alternative forms of a gene at a particular locus. In a diploid (or amphidiploid) cell of an organism, alleles of a given gene are located at a specific location or locus on a chromosome, with one allele being present on each chromosome of the pair of homologous chromosomes. Similarly, in a hexaploid cell of an organism, one allele is present on each chromosome of the group of six homologous chromosomes. “Heterozygous” alleles are different alleles residing at a specific locus, positioned individually on corresponding homologous chromosomes. “Homozygous” alleles are identical alleles residing at a specific locus, positioned individually on corresponding homologous chromosomes in the cell.

The term “globulin gene” as used herein refers to a sequence of DNA that encodes a globulin protein. A “globulin gene” also refers to alleles of globulin genes that are present at the same chromosomal position on the homologous chromosome. The term “globulin genes” refers to more than one globulin gene present within the same soybean genome. Whereas globulin genes may be different in terms of nucleotide composition, they all encode globulin proteins. A “wild type globulin gene” is a naturally occurring globulin gene (e.g., as found within naturally occurring soybean plants) that encodes a globulin protein, while a “mutant globulin gene” is a globulin gene that has incurred one or more sequence changes, where the sequence changes result in the loss, addition, or modification of amino acids within the translated protein, as compared to the wild type globulin gene. A “mutant globulin gene” can include one or more mutations in a globulin gene's nucleic acid sequence, where the mutation(s) result in the absence or reduced levels of low sulfur-containing globulin proteins in the plant or plant cell in vivo. Additionally, a “mutant globulin gene” can include a globulin gene where the full length coding sequence was deleted from the soybean genome, and are no longer capable of producing low sulfur-containing globulin protein.

The soybean genome usually contains multiple globulin genes, named Gy1-Gy8 for 11S glycinin, and Glyma10g39150, Glyma20g28650, Glyma20g28660, Glyma20g28460, and Glyma20g28640 for conglycinin genes. The methods provided herein can be used to mutate at least one (e.g., at least two, at least three, at least four, at least five, at least six, one to three, two to five, more than five, or all) globulin genes, thereby removing at least some full-length RNA transcripts and low sulfur-containing globulin protein from soybean cells, and in some cases completely removing all full-length RNA transcripts and globulin protein.

As used herein, the term “content” refers to the percentage of a certain feature among the total amount of that feature. For example, “content of a seed storage protein” refers to the percentage of that particular seed storage protein among total amount of seed storage proteins.

The term “low sulfur-containing globulin” as used herein with regard to soybean refers to seed storage proteins that are within soybean plants, cells, plant parts, and seeds that are produced from endogenous globulin genes.

Representative examples of naturally occurring soybean globulin nucleotide sequences (encoding low sulfur-containing globulin proteins) are shown in FIGS. 1A-1C (SEQ ID NOS:1, 16, and 17), FIGS. 2A-2C (SEQ ID NOS:2, 18, and 19), FIG. 3 (SEQ ID NO:3), and FIG. 4 (SEQ ID NO:4). The soybean plants, cells, plant parts, seeds, and progeny thereof that are provided herein have a mutation in one or more endogenous globulin genes, such that expression of the one or more genes is reduced or completely abolished, or the low sulfur-containing globulin protein is reduced or absent. Thus, in some cases, the plants, cells, plant parts, seeds, and progeny exhibit reduced levels of low sulfur-containing globulin.

The term “rare-cutting endonucleases” herein refer to natural or engineered proteins having endonuclease activity directed to nucleic acid sequences having a recognition sequence (target sequence) about 12-40 bp in length (e.g., 14-40, 15-36, or 16-32 bp in length). Several rare-cutting endonucleases cause cleavage inside their recognition site, leaving 4 nt staggered cuts with 3′0H or 5′0H overhangs. These rare-cutting endonucleases may be meganucleases, such as wild type or variant proteins of homing endonucleases, more particularly belonging to the dodecapeptide family (LAGLIDADG (SEQ ID NO:15); see, WO 2004/067736), or may be fusion proteins that contain a DNA binding domain and a catalytic domain with cleavage activity. TALE nucleases and zinc-finger-nucleases (ZFN) are examples of fusions of DNA binding domains with the catalytic domain of the endonuclease FokI. For a review of rare-cutting endonucleases, see Baker, Nature Methods, 9:23-26, 2012).

“Mutagenesis” as used herein refers to processes in which mutations are introduced into a selected DNA sequence. Mutations induced by endonucleases generally are obtained by a double strand break, which results in insertion/deletion mutations (“indels”) that can be detected by deep-sequencing analysis. Such mutations typically are deletions of several base pairs, and have the effect of inactivating the mutated allele. Mutations can also be introduced by generating two double-strand breaks on the same chromosome, resulting in either two indels or the deletion/inversion of intervening sequence. In the methods described herein, for example, mutagenesis occurs via double stranded DNA breaks made by TALE nucleases targeted to selected DNA sequences in a plant cell. Such mutagenesis results in “TALE nuclease-induced mutations” (e.g., TALE nuclease-induced knockouts) and reduced expression of the targeted gene, or reduced immunogenicity of the encoded protein. Following mutagenesis, plants can be regenerated from the treated cells using known techniques (e.g., planting seeds in accordance with conventional growing procedures, followed by self-pollination).

As used herein, the terms “knocking down,” “knockdown,” and “downregulation” refer to a reduction in gene expression. Downregulation of a gene can result from lower transcriptional activity or lower translational activity. Downregulation of a gene can be achieved using different technologies, including sequence-specific nucleases. Using sequence-specific nucleases, downregulation can be achieved by mutating sequences within, for example, the promoter of a gene. Without limitation, targeted mutations can be directed to the TATA box, CAAT box, GC box, proximal promoter elements, distal enhancer sequences, downstream enhancers, or other transcription factor binding sites.

As used herein, the term “complete loss of expression” refers to a complete abolition of the expression of a gene. This can include no transcriptional activity. In some cases, a complete loss of expression can be achieved using one or more sequence-specific nucleases to mutate a target sequence within the promoter of a gene.

As used herein, the terms “inactivation,” “knockout,” and “completely delete” refer to the loss of protein activity. Inactivation or knockout can occur from a frameshift mutation within a gene's coding sequence, for example. A frameshift can lead to an early stop codon and a truncated protein. A complete deletion can be obtained using one or more sequence-specific nucleases to remove all or part of a gene's coding sequence.

As used herein, “null” refers to a mutation within the coding sequence of a gene that results in the complete or near complete loss of production of the wild type protein. A “null” mutation can be a frameshift within the coding sequence of a gene, or a “null” mutation can be an in-frame deletion within the coding sequence of a gene. An in-frame deletion may result in the removal of targeted portions of a protein's amino acid sequence (e.g., an active domain or certain stretches of amino acids).

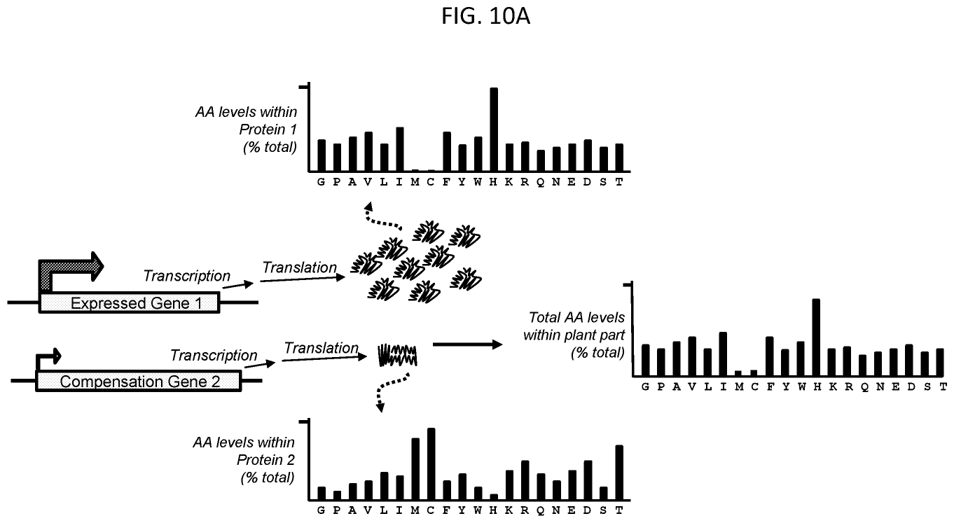

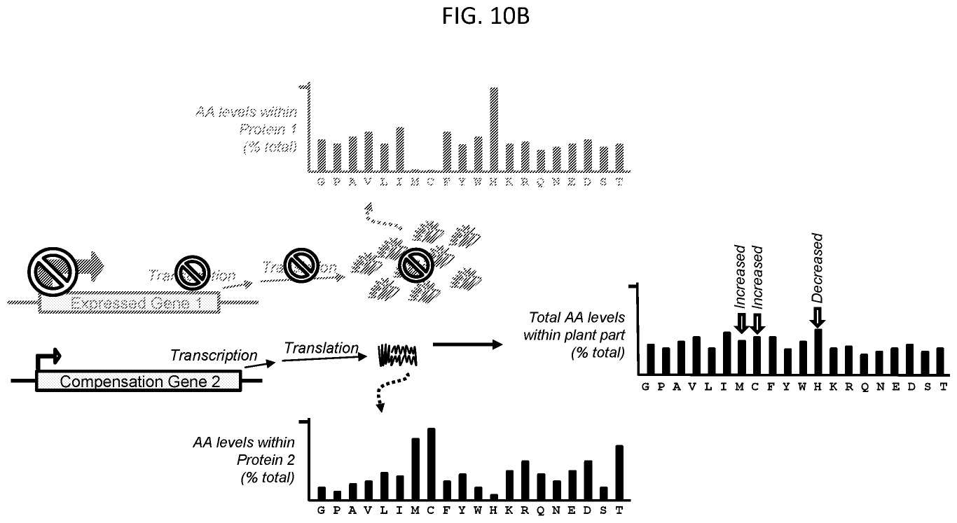

As used herein, “compensation proteins” are proteins that are encoded by compensation genes, where the compensation genes have increased expression after a different (e.g., targeted) gene is downregulated or knocked out. Compensation proteins can have a different amino acid content than the protein that is downregulated or knocked out. See, FIGS. 10A and 10B for an illustration of how compensation proteins can contribute to altering amino acid content in cells. In some embodiments, the plants, plant cells, plant parts, seeds, and progeny provided herein can be generated using a TALE nuclease system to make targeted mutations in globulin genes. Thus, this document provides materials and methods for using rare-cutting endonucleases (e.g., TALE nucleases) to generate plants (e.g., soybean plants) and related products (e.g., seeds and plant parts) that can be used as sources of protein having reduced levels of targeted proteins (e.g., soybean low sulfur-containing globulins), due to mutations in the corresponding targeted genes. Other sequence-specific nucleases also may be used to generate the desired plant material, including engineered homing endonucleases, zinc finger nucleases, and RNA-guided endonucleases.

A mutation can be, for example, a deletion (ranging from small deletions between 1 and about 100 bp, to large deletions between about 100 bp and about 100,000 bp), a substitution, or an insertion of nucleotide base pairs. In some embodiments, a mutation can be a combination of a deletion and a substitution, a deletion and an insertion, a substitution and an insertion, or a deletion, a substitution, and an insertion. In soybean, a mutation can result in inactivation of low sulfur-containing glycinin/conglycinin gene function, removal of one or more entire low sulfur-containing glycinin/conglycinin genes, and/or removal of DNA sequences that code for low sulfur-containing glycinin/conglycinin proteins. The target sequence for mutations can be within the coding sequence of Gy4 (e.g., within SEQ ID NO:1, shown in FIG. 1A), Gy5 (e.g., within SEQ ID NO:2, shown in FIG. 2A), Glyma20g28460 (e.g., within SEQ ID NO:3, shown in FIG. 3), or Glyma20g28640 (e.g., within SEQ ID NO:4, shown in FIG. 4). In some embodiments, the target sequence for a mutation can be within a coding sequence that, when translated, has at least 90% (e.g., at least 91%, at least 92%, at least 93%, at least 94%, at least 95%, at least 96%, at least 97%, at least 98%, or at least 99%) amino acid sequence identity to the sequences encoded by SEQ ID NOS:1-4 and set forth in SEQ ID NOS:5-9.

The term “expression” as used herein refers to the transcription of a particular nucleic acid sequence to produce sense or antisense RNA or mRNA, and/or the translation of an mRNA molecule to produce a polypeptide (e.g., a seeds storage protein), with or without subsequent post-translational events.

“Reducing the expression” of a gene or polypeptide in a plant or a plant cell includes inhibiting, interrupting, knocking-out, or knocking-down the gene or polypeptide, such that transcription of the gene and/or translation of the encoded polypeptide is reduced as compared to a corresponding control plant or plant cell in which expression of the gene or polypeptide is not inhibited, interrupted, knocked-out, or knocked-down. Expression levels can be measured using methods such as, for example, reverse transcription-polymerase chain reaction (RT-PCR), Northern blotting, dot-blot hybridization, in situ hybridization, nuclear run-on and/or nuclear run-off, RNase protection, or immunological and enzymatic methods such as ELISA, radioimmunoassay, and western blotting.

In general, when the plant is soybean, the soybean plant, plant part, or plant cell as provided herein can have expression of one or more globulin genes reduced by at least about 50 percent (e.g., at least about 60 percent, at least about 70 percent, at least about 80 percent, at least about 90 percent, 50 to 75 percent, or 70 to 90 percent) as compared to a corresponding control soybean plant that lacks the mutation(s). The control soybean plant can be, for example, a corresponding wild-type soybean plant in which the globulin gene(s) have not been mutated.

In some cases, a targeted nucleic acid in soybean can have a nucleotide sequence with at least about 90 percent sequence identity to a representative globulin nucleotide sequence. For example, a nucleotide sequence can have at least 90 percent, at least 91 percent, at least 92 percent, at least 93 percent, at least 94 percent, at least 95 percent, at least 96 percent, at least 97 percent, at least 98 percent, or at least 99 percent sequence identity to a representative, naturally occurring globulin nucleotide sequence.

In some cases, a mutation in soybean can be at a target sequence within a globulin coding sequence as set forth herein (e.g., SEQ ID NOS:1-4), or at a target sequence that is at least 90 percent (e.g., at least 90 percent, at least 91 percent, at least 92 percent, at least 93 percent, at least 94 percent, at least 95 percent, at least 96 percent, at least 97 percent, at least 98 percent, or at least 99 percent) identical to a globulin coding sequence as set forth herein (e.g., SEQ ID NOS:1-4), or at a target sequence that, when translated, is at least 90 percent (e.g., at least 90 percent, at least 91 percent, at least 92 percent, at least 93 percent, at least 94 percent, at least 95 percent, at least 96 percent, at least 97 percent, at least 98 percent, or at least 99 percent) identical to a globulin amino acid sequence as set forth herein (e.g., SEQ ID NOS:5-8), or at a target sequence that flanks a globulin gene and is within 100,000 bp (e.g., within 80,000 bp, within 50,000 bp, within 20,000 bp, within 20,000 to 50,000 bp, or within 50,000 to 80,000 bp) of the nearest globulin gene.

The percent sequence identity between a particular nucleic acid or amino acid sequence and a sequence referenced by a particular sequence identification number is determined as follows. First, a nucleic acid or amino acid sequence is compared to the sequence set forth in a particular sequence identification number using the BLAST 2 Sequences (Bl2seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to -1; -r is set to 2; and all other options are left at their default setting. For example, the following command can be used to generate an output file containing a comparison between two sequences: C:\Bl2seq c:\seq1.txt -j c:\seq2.txt -p blastn -o c:\output.txt -q -1 -r 2. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C: \Bl2seq c:\seq1.txt -j c:\seg2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence (e.g., SEQ ID NO:1), or by an articulated length (e.g., 100 consecutive nucleotides or amino acid residues from a sequence set forth in an identified sequence), followed by multiplying the resulting value by 100. For example, a nucleic acid sequence that has 1600 matches when aligned with the sequence set forth in SEQ ID NO:1 is 94.6 percent identical to the sequence set forth in SEQ ID NO:1 (i.e., 1600±1692×100=94.6). It is noted that the percent sequence identity value is rounded to the nearest tenth. For example, 75.11, 75.12, 75.13, and 75.14 is rounded down to 75.1, while 75.15, 75.16, 75.17, 75.18, and 75.19 is rounded up to 75.2. It also is noted that the length value will always be an integer.

Methods for selecting endogenous target sequences and generating TALE nucleases targeted to such sequences can be performed as described elsewhere. See, for example, PCT Publication No. WO 2011/072246, which is incorporated herein by reference in its entirety. In some embodiments, software that specifically identifies TALE nuclease recognition sites, such as TALE-NT 2.0 (Doyle et al., Nucleic Acids Res 40:W117-122, 2012) can be used.

Transcription activator-like effectors (TALEs) are found in plant pathogenic bacteria in the genus Xanthomonas. These proteins play important roles in disease, or trigger defense, by binding host DNA and activating effector-specific host genes (see, e.g., Gu et al., Nature 435:1122-1125, 2005; Yang et al., Proc Natl Acad Sci USA 103:10503-10508, 2006; Kay et al., Science 318:648-651, 2007; Sugio et al., Proc Natl Acad Sci USA 104:10720-10725, 2007; and Römer et al., Science 318:645-648, 2007). Specificity depends on an effector-variable number of imperfect, typically 34 amino acid repeats (Schornack et al., J Plant Physiol 163:256-272, 2006; and WO 2011/072246). Polymorphisms are present primarily at repeat positions 12 and 13, which are referred to herein as the repeat variable-diresidue (RVD).

The RVDs of TAL effectors correspond to the nucleotides in their target sites in a direct, linear fashion, one RVD to one nucleotide, with some degeneracy and no apparent context dependence. This mechanism for protein-DNA recognition enables target site prediction for new target specific TAL effectors, as well as target site selection and engineering of new TAL effectors with binding specificity for the selected sites.

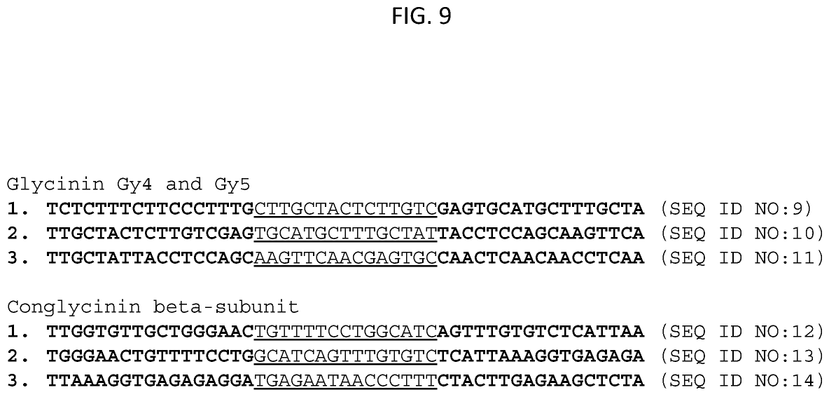

TAL effector DNA binding domains can be fused to other sequences, such as endonuclease sequences, resulting in chimeric endonucleases targeted to specific, selected DNA sequences, and leading to subsequent cutting of the DNA at or near the targeted sequences. Such cuts (i.e., double-stranded breaks) in DNA can induce mutations into the wild type DNA sequence via NHEJ or homologous recombination, for example. In some cases, TALE nucleases can be used to facilitate site directed mutagenesis in complex genomes, knocking out or otherwise altering gene function with great precision and high efficiency. As described in the Examples below, TALE nucleases targeted to the soybean globulin gene can be used to mutagenize the endogenous gene, resulting in plants without detectable expression (or reduced expression) of globulin. The fact that some endonucleases (e.g., FokI) function as dimers can be used to enhance the target specificity of the TALE nuclease. For example, in some cases a pair of TALE nuclease monomers targeted to different DNA sequences can be used. When the two TALE nuclease recognition sites are in close proximity, as depicted in FIG. 9, the inactive monomers can come together to create a functional enzyme that cleaves the DNA. By requiring DNA binding to activate the nuclease, a highly site-specific restriction enzyme can be created.

Methods for using TALE nucleases to generate plants, plant cells, or plant parts having mutations in endogenous genes include, for example, those described in the Examples herein. For example, one or more nucleic acids encoding TALE nucleases targeted to conserved nucleotide sequences present on one or more globulin genes can be transformed into plant cells or plant parts (e.g., protoplasts), where they can be expressed. In some cases, one or more TALE nuclease proteins can be introduced into plant cells or plant parts (e.g., protoplasts). The cells or plant parts, or a plant cell line or plant part generated from the cells, can subsequently be analyzed to determine whether mutations have been introduced at the target site(s), through next-generation sequencing techniques (e.g., 454 pyrosequencing or illumine sequencing). The template for sequencing can be, for example, glycinin or conglycinin genes that were amplified by PCR using primers that are homologous to conserved nucleotide sequences. Analysis of mutations can also be carried out using methods to analyze copy number (e.g., quantitative PCR [TaqMan Copy Number Assays; tools.lifetechnologies.com/content/sfs/brochures/cms 073956.pdf]). The copy number of globulin genes is analyzed because the generation of multiple double-strand breaks may lead to loss of intervening sequences, and consequently loss of multiple globulin genes.

The clustered regularly interspaced short palindromic repeats/CRISPR-associated (CRISPR/Cas) systems also can be used to direct DNA cleavage (see, e.g., Belahj et al., Plant Methods 9:39, 2013). This system consists of a Cas9 endonuclease and a guide RNA (either a complex between a CRISPR RNA [crRNA] and trans-activating crRNA [tracrRNA], or a synthetic fusion between the 3′ end of the crRNA and 5′ end of the tracrRNA). The guide RNA directs Cas9 binding and DNA cleavage to sequences that are adjacent to a proto-spacer adjacent motif (PAM; e.g., NGG for Cas9 from Streptococcus pyogenes). Once at the target DNA sequence, Cas9 generates a DNA double-strand break at a position three nucleotides from the 3′ end of the crRNA sequence that is complementary to the target sequence. As there are several PAM motifs present in the nucleotide sequence of the globulin genes, the CRISPR/Cas system may be employed to introduce mutations within the globulin alleles within soybean plant cells in which the Cas9 endonuclease and the guide RNA are transfected and expressed. This approach can be used as an alternative to TALE nucleases in some instances, to obtain plants, plant parts, and plant cells as described herein.

In some embodiments, the Cas protein can be a “functional derivative” of a naturally occurring Cas protein. A functional derivative of a native (naturally occurring) polypeptide is a compound having a qualitative biological property in common with the native polypeptide. Functional derivatives include, but are not limited to, fragments of a native polypeptide, derivatives of a native polypeptide, and derivatives of fragments of a native polypeptide, provided that the fragments and derivatives have a biological activity in common with the corresponding native polypeptide. A biological activity contemplated herein is, for example, the ability of the functional derivative to hydrolyze a DNA substrate into fragments. The term “derivative” encompasses amino acid sequence variants of a polypeptide, covalent modifications of a polypeptide, and polypeptide fusions. Suitable derivatives of a Cas polypeptide or a fragment thereof include, without limitation, mutants, fusions, covalently modified Cas polypeptides, and fragments thereof.

In some embodiments, the Cas protein can be a NmCas9, StCas9, or SaCas9 polypeptide (see, for example, Esvelt et al., Nat Methods 10:1116-1121, 2013; Steinert et al., Plant J 84:1295-1305; Kaya et al., Sci Rep 6:26871, 2016; Zhang et al., Sci Rep 7:41993, 2017; and Kaya et al., Plant Cell Physiol 58:643-649, 2017). In addition to Cas9, CRISPR systems from Prevotella and Francisella 1 (Cpf1) can be used in the methods provided herein (see, for example, Zetsche et al., Cell 163:759-771, 2015).

The invention will be further described in the following examples, which do not limit the scope of the invention described in the claims.

### EXAMPLES

### Example 1—Engineering Sequence-Specific Nucleases to Mutagenize Low Sulfur Containing Globulin Genes

To mutagenize, knock-out or completely delete low sulfur-containing globulin genes in soybean, sequence-specific nucleases were designed to target conserved nucleotides within the glycinin Gy4 (Glyma10g04280), Gy5 (Glyma13g18450), and beta-conglycinin Glyma20g28460 and Glyma20g28640 coding sequences. Target seed storage proteins were chosen based on their level of cysteine and methionine, as they contained the lowest levels of cysteine and methionine out of all the storage proteins. TABLE 1 shows the percent of methionine and cysteine in soybean seed storage proteins.

TABLE 1
Percent methionine and cysteine in soybean seed storage proteins

| | % Met and Cys |
|---|---|
| Glycinin | |
| Gy1 | 2.81% |
| Gy2 | 3.09% |
| Gy3 | 2.70% |
| Gy4 | 1.42% |
| Gy5 | 1.94% |
| Conglycinin | |
| α | 0.99% |
| α′ | 1.41% |
| β | 0.00% |

TALE nuclease target sequences were chosen within the first 200 bp of the coding sequence to increase the likelihood that a frameshift mutation will abolish the production of the targeted low sulfur-containing globulin proteins. Target sequences for TALE nuclease pairs are shown in FIG. 9. Due to sequence similarities, it is noted that the TALE nucleases targeting A3B4 may also bind to sequences within A5A4B3. TALE nucleases were synthesized using methods similar to those described elsewhere (Cermak et al., Nucleic Acids Res. 39: e82, 2011; Reyon et al., Nat Biotechnol, 30:460-465, 2012; and Zhang et al., Nat Biotechnol, 29:149-153, 2011). Individual TALE nuclease monomers were cloned into protoplast expression vectors harboring a nopaline synthase (NOS) promoter and terminator. TALE nuclease backbone architecture contained N-terminal truncations (N152: TAAAKFERQHMDSIDIADLRTLGYSQQQQEKIKPKV RSTVAQHHEALVGHGFTHAHIVALSQHPAALGTVAVKYQDMIAALPEATHEAIV GVGKQWSGARALEALLTVAGELRGPPLQLDTGQLLKIAKRGGVTAVEAVHAW RNALTGAPLN; SEQ ID NO:6401) and C-terminal truncations (C40: SIVAQLSRPDPALAALT ND HLVALACLGGRPALDAVKKGL; SEQ ID NO:6402). Repeat variable diresidues within the TALE repeats included NI (for targeting adenine), HD (for targeting cytosine), NN (for targeting guanine), and NG (for targeting thymine). To facilitate trafficking to plant cell nuclei, an SV40 NLS (PKKKRKV; SEQ ID NO:6403) was added to the N-terminus of the TALE nuclease protein.

### Example 2—Activity of TALE Nuclease Pairs at their Endogenous Target Sites in Soybean Globulin Genes

To assess TALE nuclease activity at endogenous target sequences (e.g., within Glyma10g04280, Glyma13g18450, Glyma20g28460, and/or Glyma20g28640), TALE nuclease pairs were transiently transformed into soybean protoplasts, and target sites were surveyed for mutations introduced by non-homologous end-joining (NHEJ). Transient transformation of DNA into soybean protoplasts was performed as described elsewhere (Dhir et al., Plant Cell Rep, 10: 39-43, 1991). Briefly, 15 days after pollination, immature soybean seedpods were sterilized by washing them successively in 100% ethanol, 50% bleach, and sterile distilled water. Seedpod and seed coat were removed to isolate immature seeds. Protoplasts were then isolated from immature cotyledons by enzyme digestion for 16 hours using the protocol described by Dhir et al., supra. Protoplasts were passed through a 100 μm cell filter and collected in a 50 mL Falcon tube, and were then were then pelleted by centrifugation at 100 rpm for 5 minutes. The supernatant was removed and cells were resuspended in WB-N solution (0.45 M D-mannitol, 10 mM calcium chloride, pH 5.8). Protoplasts were transformed using polyethylene glycol 4000 (20% diluted concentration) for 30 minutes. For each TALE nuclease pair, ˜500 000 protoplasts were transformed with 30 μg of plasmid (15 μg for each TALE nuclease pair). Protoplasts were washed three times in WB-N, transferred to low retention 15×10 mm petri plates, and incubated at 25° C. for 48 hours before genomic DNA was isolated using a CTAB-based method (Murray and Thompson, Nucl Acids Res, 8:4321-4325, 1980).

Using the genomic DNA prepared from the protoplasts as a template, a ˜600-bp fragment encompassing the TALE nuclease recognition site was amplified by PCR. The PCR product was then subjected to 454 pyro-sequencing. Sequencing reads with insertion/deletion (indel) mutations in the spacer region were considered to have been derived from imprecise repair of a cleaved TALE nuclease recognition site by NHEJ. Mutagenesis frequency was calculated as the number of sequencing reads with NHEJ mutations out of the total sequencing reads. The values were then normalized by the transformation efficiency (82%, as determined by a YFP-expression control plasmid). A summary of the TALE nuclease mutagenesis frequencies is shown in TABLE 2. Mutations introduced into soybean cells by the GmBCG2_T01 TALE nuclease pairs are listed in SEQ ID NOS:23-149. Mutations introduced by the GmBCG2_T02 TALE nuclease pairs are listed in SEQ ID NOS:150-475. Mutations introduced with the GmBCG2 T03 TALE nuclease pairs are listed in SEQ ID NOS:476-506. Mutations introduced by the GmGlyA3B4_T01 TALE nuclease pairs are listed in SEQ ID NOS:507-1688. Mutations introduced by the GmGlyA3B4_T02 TALE nuclease pairs are listed in SEQ ID NOS:1689-4768. Mutations introduced into soybean cells with the GmGlyA3B4_T03 TALE nuclease pairs are listed in SEQ ID NOS:4769-6347. SEQ ID NOS:23-6347 are shown in the attached Sequence Listing.

TABLE 2
Summary of GmBCG2 and GmGlyA3B4 TALE endonuclease activity in soybean protoplasts

| | | | Raw | Normalized |
|---|---|---|---|---|
| | | | mutation | mutation |
| | | | frequency | frequency |
| Target | Name | Target sequence | (%) | (%) |
| Glycinin | GmGlyA3B4_T01 | TCTCTTTCTTCCCTTTGCTTGCTACTCTTGTCG | 9.62 | 11.73 |
| | | AGTGCATGCTTTGCTA (SEQ ID NO: 9) | | |
| | GmGlyA3B4_T02 | TTGCTACTCTTGTCGAGTGCATGCTTTGCTATT | 25.22 | 30.76 |
| | | ACCTCCAGCAAGTTCA (SEQ ID NO: 10) | | |
| | GmGlyA3B4_T03 | TTGCTATTACCTCCAGCAAGTTCAACGAGTGCC | 13.05 | 15.91 |
| | | AACTCAACAACCTCAA (SEQ ID NO: 11) | | |
| Conglycinin | GmBCG2_T01 | TTGGTGTTGCTGGGAACTGTTTTCCTGGCATCA | 1.7 | 2.1 |
| beta-subunit | | GTTTGTGTCTCATTAA (SEQ ID NO: 12) | | |
| | GmBCG2_T02 | TGGGAACTGTTTTCCTGGCATCAGTTTGTGTCT | 4.58 | 5.59 |
| | | CATTAAAGGTGAGAGA (SEQ ID NO: 13) | | |
| | GmBCG2_T03 | TTAAAGGTGAGAGAGGATGAGAATAACCCTTTC | 3.44 | 4.2 |
| | | TACTTGAGAAGCTCTA (SEQ ID NO: 14) | | |

### Example 3—Regeneration of Soybean Lines with TALE Nuclease-Induced Mutations in Low Sulfur-Containing Globulin Genes

TALE nucleases showing activity were then used to create soybean lines with mutations in glycinin genes. Toward that end, the GmGlyA3B4_T02 TAL effector endonuclease pair was cloned into a bacterial vector, with TALE nuclease expression driven by the cauliflower mosaic virus 35S promoter. Following transformation of soybean half cotyledons (variety Bert) with sequences encoding the GmGlyA3B4_T02 TAL effector endonuclease, candidate transgenic plants (into which the GmGlyA3B4_T02 TAL effector endonuclease sequences were genomically integrated) were regenerated. The plants were transferred to soil, and after about 4 weeks of growth, a small leaf was harvested from each plant for DNA extraction and genotyping. Transgenic T0 individuals were assayed by PCR of the target locus (GlyA3B4) and subsequent direct Sanger sequencing of the PCR product. Sequencing traces that contained disruptions at or near the center of the target site were considered to be mutant. The original PCR product was then cloned into a pJet vector for individual genotype characterization.

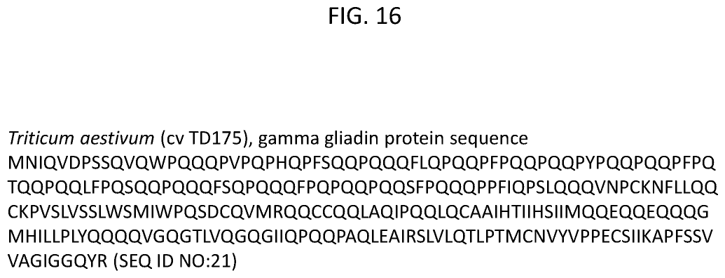

One shoot (Gm318-1) was observed with mutations at the GlyA3B4 locus. A summary of the transformation experiments are shown in TABLE 3. Seed from the Gm318-1 plant was collected and grown into T1 plants. Genomic DNA from T1 plants was isolated and the GlyA3B4 and GlyA5A4B3 and TALE nuclease target site were sequenced. Deletions within both of the GlyA3B4 and GlyA5A4B3 target sites were observed within T1 plants. Examples of the mutations are shown in FIGS. 16A and 16B.

Tissue from T2 seeds was collected for analysis of mutations at the glycinin loci. Toward that end, 715 T1 seeds were collected from the T1 plants Gm318-1-1, Gm318-1-2, Gm318-1-3, and Gm318-1-4. The seeds were germinated in a greenhouse in a soil mixture in under 30° C./27° C. (16 hour day/8 hour night) with 65% humidity. The germination frequency was 80.2%. Two weeks after germination, leaf samples were collected from individual T2 plants and DNA was extracted. The DNA was tested for the presence of the TALE nuclease DNA and for mutations at the Gy4 and Gy5 glycinin loci. Primers used for amplifying the GmGlyA3B4_T02 binding site in the GlyA3B4 and GlyA5A4B3 genes are shown in TABLE 4.

TABLE 3
Summary of transformation experiments using the GmGlyA3B4_T02
nuclease pair

| | | | Number of shoots |
|---|---|---|---|
| Experiment | Number of explants | Number of | mutant at the |
| name | transformed | transgenic shoots | GlyA3B4 locus |
| Gm318 | 120 | 1 | 1 |
| Gm319 | 147 | 1 | 0 |
| Gm326 | 159 | 0 | 0 |
| Gm327 | 136 | 0 | 0 |
| Gm449 | 114 | 0 | 0 |
| Gm450 | 100 | 0 | 0 |
| Gm452 | 100 | 0 | 0 |
| Gm486 | 92 | 0 | 0 |
| Gm516 | 87 | 0 | 0 |
| Gm518 | 60 | 0 | 0 |
| Gm536 | 48 | 0 | 0 |
| Gm537 | 84 | 0 | 0 |
| Gm541 | 72 | 0 | 0 |
| Gm560 | 96 | 0 | 0 |
| Gm578 | 90 | 0 | 0 |
| Gm579 | 86 | 0 | 0 |
| Gm582 | 78 | 0 | 0 |
| Gm584 | 91 | 0 | 0 |
| Gm606 | 96 | 0 | 0 |
| Gm608 | 93 | 0 | 0 |
| Gm611 | 144 | 0 | 0 |
| Gm619 | 90 | 0 | 0 |
| Gm621 | 96 | 0 | 0 |
| Gm624 | 96 | 5 | 0 |

TABLE 4
Primers for amplifying the GmGlyA3B4_T02 binding
site in the GlyA3B4 and GlyA5A4B3 genes

| | Target | | SEQ |
|---|---|---|---|
| Primer Name | Gene | Sequence | ID: |
| CLXGmGLY3i1F | GlyA3B4 | TTCACTATAAATCGCCACTCT | 6348 |
| | (Gy5) | TCG | |
| CLXGmGLY3i2R | GlyA3B4 | CTAATATTACGCACCTTGAAC | 6349 |
| | (Gy5) | GACA | |
| CLXGmGLY504H | GlyA5A4B3 | ACCACTCCTCATGTTCTTTCC | 6350 |
| | (Gy4) | AA | |
| CLXGmGLY505H | GlyA5A4B3 | GTTGAGAGTTCCATGTTTGAA | 6351 |
| | (Gy4) | TCAA | |

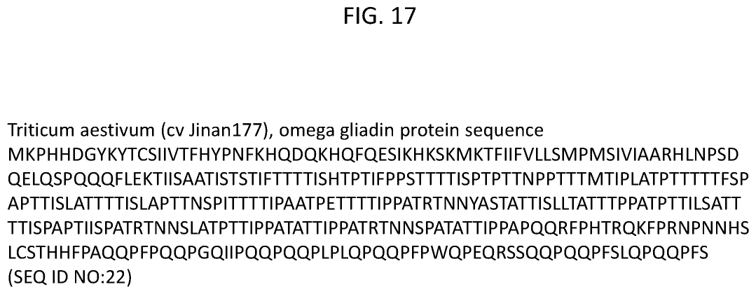

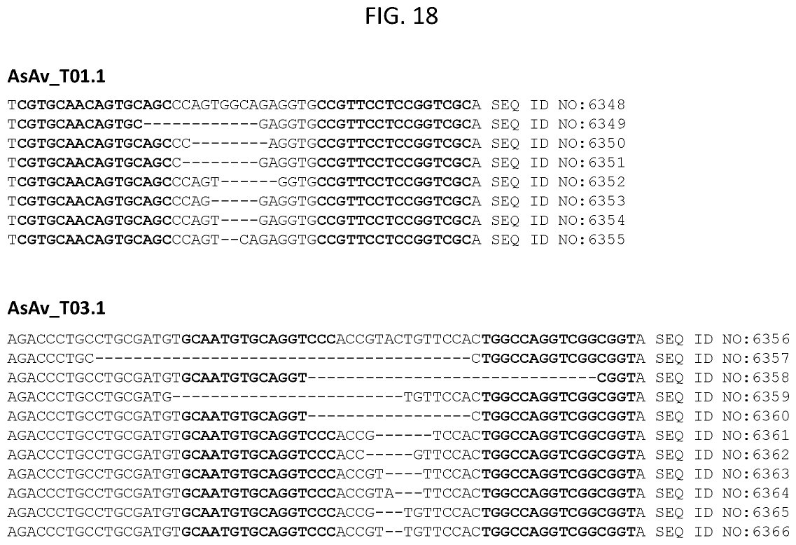

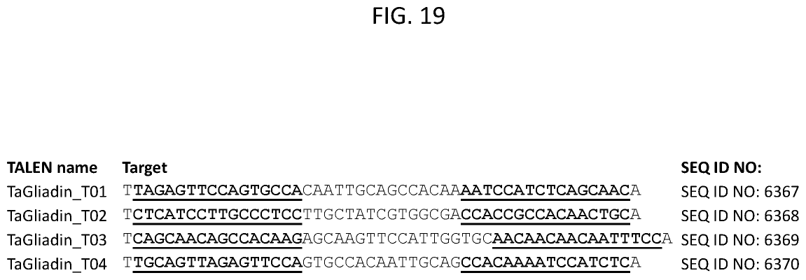

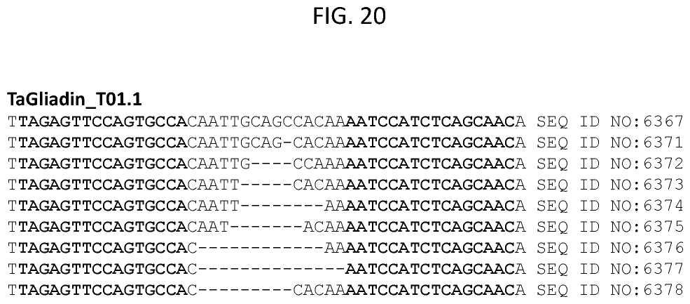

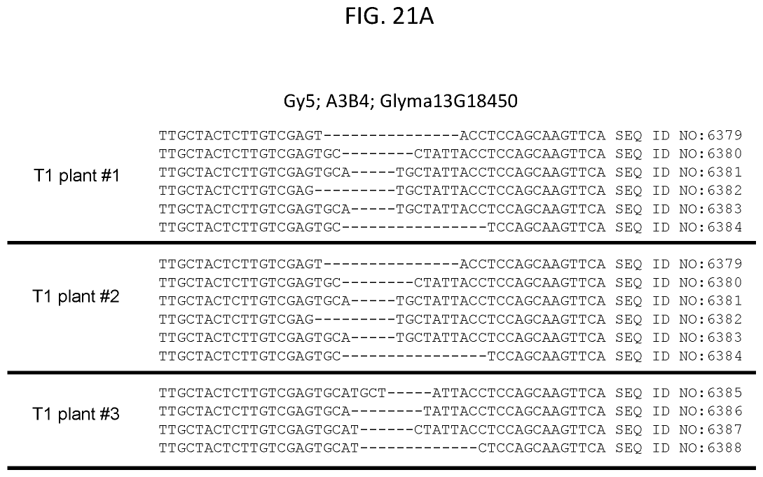

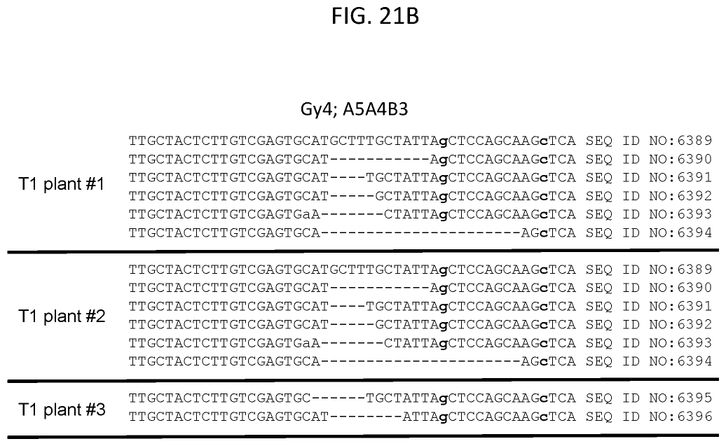

Mutations identified in the Gy4 and Gy5 genes in a T2 plant from the parent Gm318-1-4 are shown in FIG. 17. Mutations identified in the Gy4 and Gy5 genes in T2 plant 1, plant 2, and plant 3 from the parent Gm318-1-2 are shown in FIGS. 18, 19, and 20, respectively.

### Example 4—Assessing the Phenotype of Modified Soybean Plants

Soybean plants containing mutations within low sulfur-containing globulin genes were assessed for low sulfur-containing globulin content. Initial screening to identify seeds with altered globulin content is performed by one-dimensional SDS-PAGE in which total soluble protein is stained with 0.1% Coomassie Brilliant Blue, and a replicate immunoblot is probed using a mixture of polyclonal antibodies, one specific to glycinin and another to beta-conglycinin as described elsewhere (Schmidt et al. 2011, supra). Non-transformed soybean seed is used as a positive control. Seeds whose corresponding protein profiles are shown to have the desired phenotype, namely a reduction in low sulfur-containing globulin proteins and an increase in high sulfur-containing globulins, are grown into the next generation. Two generations may be grown and screened in this manner, until homozygosity is obtained.

Secondary screening to identify seeds with a change in protein composition is performed by two-dimensional protein analysis and mass spectroscopy. Total soluble protein is isolated from mature seeds as described elsewhere (Schmidt and Herman, Plant Biotech J, 6:832-842, 2008). Soluble protein extracts (150 mg) from both a non-transformed soybean seed and a homozygous globulin knock-out seed are separated in the first dimension on 11-cm immobilized pH gradient gel strips (pH 3-10 nonlinear; Bio-Rad) and then in the second dimension by SDS-PAGE gels (8%-16% linear gradient). The resulting gels are subsequently stained with 0.1% (w/v) Coomassie Brilliant Blue R250 in 40% (v/v) methanol, 10% (v/v) acetic acid overnight, and then destained for about 3 hours in 40% methanol, 10% acetic acid. Individual spots of interest are excised and digested with trypsin, and the fragments are analyzed and identified by tandem mass spectroscopy as described elsewhere (Schmidt and Herman, Mol Plant, 1:910-924, 2008). Mass spectroscopy is used to establish the identity of the proteins that are changing in abundance in the mutant seed, making it possible to definitively identify mutant soybean lines with lower levels of low sulfur-containing proteins. Overall levels of methionine and cysteine in the mutant seed are determined by quantitation of hydrolyzed amino acids and free amino acids using a Waters Acquity ultraperformance liquid chromatography system (Schmidt et al. 2011, supra).

Seeds from four T2 plants with complete knockout of the Gy4 and Gy5 genes were collected and analyzed for amino acid content, which was determined using AOAC official methods 988.15 (tryptophan), 994.12 (cystine and methionine), and 982.30 (amino acids). Controls 1-3 were seed from Glycine max plants not containing mutations in the Gy4 and Gy5 genes. Cystine content in the Gy4 and Gy5 knockout lines was 1.48%, and methionine content was 1.42% (TABLE 5). Cystine content in the three control lines was 1.29%, 1.30%, and 1.28%, and methionine content was 1.29%, 1.28%, and 1.31%.

TABLE 5
Percentage of amino acids in soybean seeds with Gy4 and Gy5 knockout
mutations

| | Control 1 | Control 2 | Control 3 | Gy4 Gy5 KO |
|---|---|---|---|---|
| Tryptophan | 1.37 | 1.33 | 1.34 | 1.48 |
| Cystine | 1.29 | 1.30 | 1.28 | 1.48 |
| Methionine | 1.29 | 1.28 | 1.31 | 1.42 |
| Alanine | 3.75 | 3.76 | 3.82 | 3.79 |
| Arginine | 6.35 | 6.84 | 6.38 | 6.16 |
| Aspartic Acid | 10.37 | 10.85 | 10.39 | 10.31 |
| Glutamic Acid | 15.97 | 16.97 | 16.11 | 15.40 |
| Glycine | 3.91 | 3.96 | 3.95 | 4.03 |
| Histidine | 2.38 | 2.43 | 2.40 | 2.55 |
| Isoleucine | 4.13 | 4.24 | 4.17 | 4.32 |
| Leucine | 6.84 | 7.04 | 6.86 | 6.93 |
| Phenylalanine | 4.54 | 4.69 | 4.54 | 4.50 |
| Proline | 4.46 | 4.66 | 4.57 | 4.38 |
| Serine | 4.65 | 4.86 | 4.67 | 4.68 |
| Threonine | 3.64 | 3.68 | 3.66 | 3.55 |
| Total Lysine | 6.32 | 6.04 | 6.01 | 5.63 |
| Tyrosine | 3.17 | 3.18 | 3.18 | 3.26 |
| Valine | 4.38 | 4.41 | 4.35 | 4.50 |

### Example 5—Designing TALE Nucleases Targeted to Low-Lysine Alpha-Gliadin Genes in Wheat

To identify the genomic sequences of alpha-gliadin genes, alpha-gliadin DNA and mRNA sequences were downloaded from NCBI and aligned. In total, 315 sequences were aligned and used to identify semi-conserved regions for primer design. Two primers were designed to amplify a ˜365 bp sequence from the 5′ end of the alpha gliadin genes.

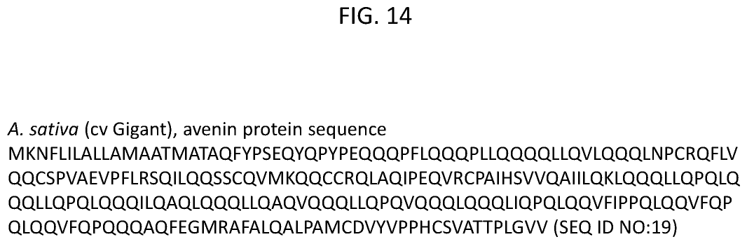

The alpha-gliadin genes were resequenced within Bobwhite 208, CPAN1796 and Chinese81. Using these sequences, TALE nucleases were designed to target sites within the 5′ end of alpha-gliadin genes, near the start codon. TALE nuclease design was performed manually. Target sequences were chosen either within semi-conserved regions (such that the TALE nucleases would bind to the majority of alpha-gliadin genes) or within divergent sequences (such that the TALE nucleases would bind to a subset of alpha-gliadin genes). With respect to designing TALE nucleases targeted to semi-conserved sequences, it is noted that there were no regions of about 50 nt that were conserved between the different alpha gliadin genes, but there were many instances in which a degenerate RVD could be used to maximize the number of TALE nuclease target sites. For example, two genes having several G or A SNPs could be targeted by designing a TALE nuclease with an NN RVD, since NN binds to both G and A. This strategy was used to design TALE nucleases TaGliadin_T01.1, TaGliadin_T02.1, and TaGliadin_T03.1. Notably, TALE nuclease TaGliadin_T02.1 contained an N* RVD to facilitate binding to all four nucleotides. To design TALE nuclease pairs that target only a subset of alpha-gliadin genes, the binding preference of TALE nucleases to T at the -1 position was exploited. Using this strategy, a fourth TALE nuclease pair (TaGliadin_T04.1) was designed. This pair was predicted to bind to a minority of alpha-gliadin genes. The TaGliadin TALE nuclease target sequences are shown in FIG. 14.

### Example 6—Transformation of Wheat Protoplasts and Use of Chemicals to Increase Mutation Frequencies

To assess the activity of alpha-gliadin TALE nuclease pairs, wheat protoplasts were isolated and transformed with 15 ug of each TALE nuclease plasmid. As a control for transformation efficiency, protoplasts were transformed with 20 ug of a YFP-expression plasmid (pNOS:YFP). For each experimental sample, about 200,000 protoplasts were transformed using polyethylene glycol.

To carry out these studies, wheat seeds were sown on MS medium and placed in a growth incubator at 25° C. with a 16 hour light/8 hour dark cycle. Protoplasts were collected from forty 14 day-old seedlings, as follows. Seedlings were removed from the medium (without roots) and cut horizontally into ˜1-2 mm sections. Tissue was placed in digestion solution (1.5% cellulase R10, 0.75% macerozyme R10, 0.6 M mannitol, 10 mM MES pH 5.7, 10 mM CaCl₂, and 0.1% BSA) and moved to a 25° C. incubator. The digestion mixture was kept in the dark for 6-7 hours with shaking at 25 rpm. Following digestion, protoplasts were isolated using methods described elsewhere (Shan et al., Nature Biotechnol 31:686-688, 2013).

Protoplasts (˜200,000) were transformed with 15 ug each of plasmids encoding TALE nuclease pairs TaGliadin_T01.1, TaGliadin_T02.1, TaGliadin_T03.1, and TaGliadin_T04.1. Protoplasts also were transformed with a 35S:YFP control to measure transformation efficiency. Following transformation, protoplasts were incubated at 25° C. in the dark for 48 hours. Protoplasts were then pelleted by centrifugation, and DNA was isolated. PCR was conducted to amplify sequences encompassing the TALE nuclease binding sites, and the resulting amplicons were deep sequenced.

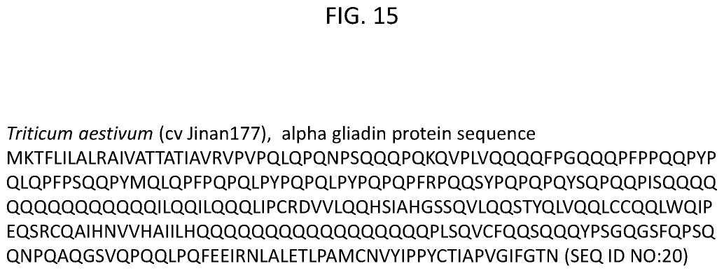

To determine the activity of each TALE nuclease pair at its target sequence, genomic DNA was isolated from protoplasts ˜48 hours post transformation, and amplicons encompassing the T1, T2, T3, and T4 target sites were generated by PCR and then deep sequenced using 454 pyrosequencing. Results from the deep sequencing analysis are shown in TABLE 6. Mutations were observed in samples for the TaGliadin_T01.1 and T02.1 TALE endonuclease pairs. Specifically, TALE nuclease pair TaGliadin_T01.1 had 0.325% activity, and TaGliadin_T02.1 had 0.746% activity. TALE nuclease pairs TaGliadin_T03.1 and TaGliadin_T04.1 had 0% activity. FIG. 15 shows examples of mutations identified in wheat protoplasts after delivery of the TaGliadin_T01.1 TALE nuclease pair.

TABLE 6
TALE nuclease mutation frequencies within alpha gliadin genes
in wheat protoplasts

| TALE nuclease | Transformation | | Mutation |
|---|---|---|---|
| constructs | Frequency | Experiment number | Frequency (%) |
| TaGliadin_T01.1 | 76.90% | Ta066 | 0.325 |
| TaGliadin_T02.1 | 76.90% | Ta067 | 0.746 |
| TaGliadin_T03.1 | 76.90% | Ta068 | 0 |
| TaGliadin_T04.1 | 76.90% | Ta069 | 0 |

In an effort to increase the frequency of mutations at the alpha-gliadin genes, the protoplast transformation was repeated three additional times using different treatments in the three transformations. In the first study, wheat protoplasts were transformed with or without a plasmid encoding TREX, which may facilitate imprecise DNA repair at the alpha-gliadin target sequences. In the second study, wheat seedlings were germinated and grown on medium containing 20 uM of 5-azacytidine. After 9 days of growth, the resulting seedlings were used for protoplast isolation and transformation, to determine whether the passive demethylation of alpha-gliadin genes using 5-azacytidine would allow TALE endonucleases to better recognize and cleave their target sequences. In the third study, wheat seedlings were germinated and grown on medium containing 4 uM of trichostatin A, which selectively inhibits histone deacetylase families of enzymes. If the heterochromatic state of alpha-gliadin genes prevents TALE endonuclease binding and cleavage, the addition of trichostatin A may relax the chromatin and allow access to the alpha-gliadin target sequences.

Results from 454 deep sequencing are shown in TABLE 7. TaGliadin_T01.1 had mutation frequencies of 1.57%, 2.40%, and 1.29% with delivery of TALE nuclease only, co-delivery of TREX, and treatment with 5-azacytidine, respectively. Further, it was observed that TaGliadin_T02.1 had the highest mutation frequency, reaching over 5% when delivered to protoplasts derived from plants treated with 5-azacytidine. See, TABLE 7 for a summary of the mutation frequencies.

### Example 7—Regeneration and Phenotyping of Wheat Lines with TALE Nuclease-Induced Mutations in Low-Lysine Containing Gliadin Wheat Genes

Functional TALE nuclease pairs are stably integrated into the wheat genome using standard transformation methods (Sparks et al., Methods Mol Biol. 478:71-92, 2009 and Jones et al., Plant Methods 1, 2005). Transgenic wheat plants are screened for mutations at the alpha-gliadin target sequences. Plants harboring mutations within the alpha-gliadin genes are advanced to phenotyping.

Initial screening to identify seeds with altered gliadin content is performed by one-dimensional SDS-PAGE in which total soluble protein is stained with 0.1% Coomassie Brilliant Blue, and a replicate immunoblot is probed using antibodies against gliadin protein. A decrease in the amount of low-lysine gliadin proteins indicates the successful reduction of protein with undesired amino acids.

Secondary screening to identify seeds with a change in protein composition is performed by two-dimensional protein analysis and mass spectroscopy. Total soluble protein is isolated from mature seeds as described elsewhere (Schmidt and Herman, Plant Biotech J, 6:832-842, 2008). Soluble protein extracts (150 mg) from both a non-transformed wheat seed and a homozygous gliadin knock-out seed are separated in the first dimension on 11-cm immobilized pH gradient gel strips (pH 3-10 nonlinear; Bio-Rad) and then in the second dimension by SDS-PAGE gels (8%-16% linear gradient). The resulting gels are subsequently stained with 0.1% (w/v) Coomassie Brilliant Blue R250 in 40% (v/v) methanol, 10% (v/v) acetic acid overnight, and then destained for about 3 hours in 40% methanol, 10% acetic acid. Individual spots of interest are excised and digested with trypsin, and the fragments are analyzed and identified by tandem mass spectroscopy as described elsewhere (Schmidt and Herman, Mol Plant, 1:910-924, 2008). Mass spectroscopy is used to establish the identity of the proteins that are changed in abundance in the mutant seed, making it possible to definitively identify mutant wheat lines with lower levels of low lysine-containing proteins. Overall levels of lysine in the mutant seed are determined by quantitation of hydrolyzed amino acids and free amino acids using a Waters Acquity ultraperformance liquid chromatography system (Schmidt et al. 2011, supra).

TABLE 7
TALE nuclease mutation frequencies within alpha gliadin genes in wheat protoplasts

| | | | | | | Mutation |
|---|---|---|---|---|---|---|
| TALE nuclease | | Transformation | Experiment | Total Reads | Total Reads | frequency |
| constructs | Treatment | Frequency | number | Analyzed | with Deletions | (%) |
| TaGliadin_T01.1 | Conventional (TALE | 72.10% | Ta081 | 3060 | 34 | 1.57 |
| | nucleases only) | | | | | |
| | TREX | 72.10% | Ta077 | 7734 | 133 | 2.40 |
| | 5-Azacytidine | 59.89% | Ta106 | 6060 | 46 | 1.29 |
| | Trichostatin A | 88.09% | Ta113 | 6527 | 51 | 0.90 |
| TaGliadin_T02.1 | Conventional (TALE | 72.10% | Ta082 | 2451 | 0 | 0.00 |
| | nucleases only) | | | | | |
| | TREX | 72.10% | Ta078 | 10591 | 178 | 2.43 |
| | 5-Azacytidine | 59.89% | Ta107 | 17697 | 552 | 5.33 |
| | Trichostatin A | 88.09% | Ta114 | 6215 | 0 | 0.00 |
| TaGliadin_T03.1 | Conventional (TALE | 72.10% | Ta083 | 5298 | 0 | 0.00 |
| | nucleases only) | | | | | |
| | TREX | 72.10% | Ta079 | 7785 | 0 | 0.00 |
| | 5-Azacytidine | 59.89% | Ta108 | 3640 | 0 | 0.00 |
| | Trichostatin A | 88.09% | Ta115 | 8522 | 0 | 0.00 |
| TaGliadin_T04.1 | Conventional (TALE | 72.10% | Ta084 | 1211 | 0 | 0.00 |
| | nucleases only) | | | | | |
| | TREX | 72.10% | Ta080 | 4206 | 0 | 0.00 |
| | 5-Azacytidine | 59.89% | Ta109 | 3370 | 0 | 0.00 |
| | Trichostatin A | 88.09% | Ta116 | 12455 | 91 | 0.86 |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A plant, plant part, or plant cell comprising a mutation in at least one seed storage protein gene that is endogenous to the plant, plant part, or plant cell, wherein the plant, plant part, or plant cell has altered amino acid content as compared to a control plant, plant part or plant cell that lacks the mutation, wherein the mutation was introduced using a rare-cutting endonuclease, and wherein the mutation is a deletion of one or more base pairs.

2. (canceled)

3. The plant, plant part, or plant cell of claim 2, wherein the rare-cutting endonuclease is a transcription activator-like effector (TALE) nuclease, meganuclease, zinc finger nuclease (ZFN), or clustered regularly interspaced short palindromic repeat (CRISPR)/Cas reagent.

4. The plant, plant part, or plant cell of claim 1, wherein the at least one seed storage protein gene is selected from the group consisting of a glycinin gene, a beta-conglycinin gene, a glutenin gene, a gliadin gene, a zein gene, a hordein gene, a secalin gene, and a prolamine gene.

5-8. (canceled)

9. The plant, plant part or plant cell of claim 1, wherein the at least one seed storage protein gene comprises a Gy4 gene, a Gy5 gene, or a beta-conglycinin gene.

10. (canceled)

11. The plant, plant part, or plant cell of claim 9, wherein the altered amino acid content comprises an increase in methionine or cysteine content as compared to a corresponding control plant, plant part, or plant cell that lacks the mutation.

12. The plant, plant part or plant cell of claim 1, wherein the at least one seed storage protein gene comprises an alpha-gliadin gene, an omega-gliadin gene, or a gamma-gliadin gene.

13. (canceled)

14. The plant, plant part, or plant cell of claim 12, wherein the altered amino acid content comprises an increase in lysine content as compared to a corresponding control plant, plant part, or plant cell that lacks the mutation.

15. A method for making a plant having altered amino acid content, comprising:
(a) contacting plant cells or plant parts comprising functional seed storage protein genes with a rare-cutting endonuclease targeted to a sequence within one or more of the functional seed storage protein genes, or to a sequence flanking the functional seed storage protein genes;
(b) growing the contacted plant cells or plant parts into plants; and
(c) selecting, from the plants, a plant with a mutation in at least one seed storage protein gene, wherein the mutation is a deletion of one or more base pairs.

16. The method of claim 15, wherein the rare-cutting endonuclease is a TALE nuclease, meganuclease, ZFN, or CRISPR/Cas reagent.

17. The method of claim 15, wherein the at least one seed storage protein gene is selected from the group consisting of a glycinin gene, a beta-conglycinin gene, a glutenin gene, a gliadin gene, a zein gene, a hordein gene, a secalin gene, and a prolamine gene.

18-21. (canceled)

22. The method of claim 15, wherein the at least one seed storage protein gene comprises a Gy4 gene, a Gy5 gene, or a beta-conglycinin gene.

23. (canceled)

24. The method of claim 22, wherein the altered amino acid content comprises an increase in methionine or cysteine content as compared to a corresponding control plant that lacks the mutation.

25. The method of claim 15, wherein the at least one seed storage protein gene comprises an alpha-gliadin gene, an omega-gliadin gene, or a gamma-gliadin gene.

26. (canceled)

27. The method of claim 25, wherein the altered amino acid content comprises an increase in lysine content as compared to a corresponding control plant, plant part, or plant cell that lacks the mutation.

28. A method for mutagenizing a cell, comprising:
(a) treating the cell with an agent that reduces DNA methylation or interferes with histone deacetylase activity; and
(b) contacting the cell with a rare-cutting endonuclease.

29. The method of claim 28, wherein the cell is a plant cell.

30. The method of claim 28, wherein the chemical is 5-azacytidine or trichostatin A.

31. The method of claim 28, wherein the rare-cutting endonuclease is a TALE nuclease, meganuclease, ZFN, or CRISPR/Cas reagent.
